(19) United States
(12) Patent Application Publication
(10) Pub. No.: US 2003/0104386 A1
(43) Pub. Date: Jun. 5, 2003

(54) Methods for the specific detection of redox-active tags and the use thereof for capillary gel electrophoresis and DNA sequencing

(75) Inventors: Werner G. Kuhr, Oak Hills, CA (US); Sara A. Brazill, Diamond Bar, CA (US)

Correspondence Address: Patrick G. Burns, Esq., Greer, Burns & Crain, Ltd., 300 So. Wacker Drive, Suite 2500, Chicago, IL, 60606, US

(73) Assignee: The Regents of the University of California

(21) Appl. No.: 09/945,238

(22) Filed: Aug. 31, 2001

(51) Int. Cl. C12Q 1/68; C12Q 1/00

(52) U.S. Cl. 435/6; 205/777.5

(57) ABSTRACT

This invention provides novel approach to the specific detection of redox-active moieties (e.g.) in a population of redox-active moieties. In particular this invention provides a “phase-nulling” technique that can be used in the electrochemical detection of redox-active tags. The signal for each tag is selectively eliminated while the other tag's response remains virtually unchanged. This novel analysis scheme allows for the simple identification of a tag of interest in a complex matrix and is demonstrated with both flow injection analysis and capillary gel electrophoresis.

### STATEMENT AS TO RIGHTS TO INVENTIONS MADE UNDER FEDERALLY SPONSORED RESEARCH AND DEVELOPMENT

This work was supported by the National Institutes of Health Grant 1R21HG01828-01. The Government of the United States of America may have certain rights in this invention.

### CROSS-REFERENCE TO RELATED APPLICATIONS

[Not Applicable]

### FIELD OF THE INVENTION

This invention pertain to the field of electrochemistry. In particular, this invention pertains to the discovery of a method of selectively detecting a redox-active moiety in a mixture of redox-active moieties.

### BACKGROUND OF THE INVENTION

The separation, detection and elucidation of DNA fragments are essential to many research areas including; DNA sequencing, forensic fingerprint analysis, disease diagnosis and drug design. The completion of a “rough draft” of the human genome announced on Jun. 26, 2000 is arguably one of the most impressive scientific achievements to date. The initial sequencing, however, is only the beginning. Currently, there are ongoing efforts to re-sequence the human genome, identify single nucleotide polymorphism's (SNP's), and sequence the genome of other organisms (Kennedy (2001) Science 291: 1153-1207). Sequencing technology has improved orders of magnitude from what it was at the start of the human genome project (1989) however, there is still great need for additional advancements to further reduce costs and increase the throughput of sequencing analysis.

The use of capillary gel electrophoresis (CGE) is widely recognized as a powerful tool in DNA sequencing and fragment analysis (Smith (1991) Nature 349: 812-813; Luckey et al. (1990) Nucleic Acids Research, 18: 4417-4421; Carrilho (2000) Electrophoresis, 21: 55-65). Traditionally, DNA separations were performed in a slab gel format with the use of either radio or fluorescent labels. More recently, CGE has become the method of choice for DNA sequencing and fingerprinting because it offers reduced run times, higher efficiency, less sample consumption and is compatible with automated sample injection (Lu et al. (1994) J. Chromatogr. A, 680: 503-510; Luckey and Smith (1993) Anal. Chem., 65: 2841-2850; Yan et al. (1996) J. Electrophoresis, 17: 1037-1045; Swerdlow and Gesteland (1990) Nucleic Acids Research, 18: 1415-1419). Replaceable sieving matrices have been developed and optimized to separate and sequence oligonucleotides with greater ease and reproducibility (Sunada and Blanch (1997) Electrophoresis, 18: 2243-2254). Entangled polymers like linear polyacrylamide (Ruiz-Martinez et al. (1993) Anal. Chem., 65: 2851-2858; Zhang et al. (1995) J. Anal. Chem., 67: 4589-4593; Carrilho et al. (1996) Anal. Chem., 68: 3305-3313), poly(vinylpyrrolidone) (Gao and Yeung (1998) Anal. Chem., 70: 1382-1388), poly(ethylene oxide) (Fung and Yeung (1995) Anal. Chem., 67: 1913-1919), and polydimethyl-acrylamide (Rosenblum et al. (1997) Nucleic Acids Research, 25: 3925-3929; Madabhushi (1998) Electrophoresis, 19: 224-230) are some examples of the polymer systems that are utilized for DNA separation. In particular, the polydimethyl-acrylamide (Id.) system is available commercially from Applied Biosystems, as the performance optimized polymer (POP) product line and is utilized in the work presented here. The development of sieving matrices with properties that achieve the maximum resolution and length of read for DNA sequencing is still an exciting area of research (Muller et al. (1998) Electrophoresis, 19: 1436-1444; Barron et al. (1996) Electrophoresis, 17: 744-757; Luckey et al. (1993) J. Phys. Chem., 97: 3067-3075).

Laser induced fluorescence (LIF) is almost exclusively employed for the detection of DNA sequencing ladders, primarily due to the development of four-color sequencing methods by Hood and co-workers (Smith et al. (1986) Nature, 321: 674-679; Smith et al. (1987) Meth. Enzymol., 155: 260-301). In four-color sequencing, each base terminated set of ladders from a Sanger dideoxy chain-termination reaction is tagged with a unique fluorophore. The primary limitation in this approach is that it is difficult to find four different fluorophores that have high molar absorption at a single wavelength, so often two wavelengths are needed for excitation. To overcome this limitation, Mathies et al. developed energy-transfer (ET) dyes that contain a common donor fluorophore and four unique acceptors that are situated an optimum distance from the donor (Ju et al. (1995) Proc. Natl. Acad. Sci., USA, 92: 4347-4351; Ju, J. Y.; Glazer, A. N.; Mathies, R. A. Nature Medicine 1996, 2, 246-249). The advantage of ET dyes is the ability to equally excite all four tags with a single wavelength (488 nm). Fluorescence lifetime measurements have also been proposed as an alternative detection strategy in DNA sequencing (Soper, S. A.; Legendre, B. L.; Williams, D. C. Anal. Chem. 1995, 67, 4358-4365; He, H.; Nunnally, B. K.; Li, L. C.; McGown, L. B. Anal. Chem. 1998, 70, 3413-3418; Lieberwirth et al. (1998) J. Anal. Chem., 70: 4771-4779). In this case, the lifetime of the four dyes is used as the discriminating characteristic rather than fluorescence emission.

Although all of these methods have proven to be both sensitive and amenable to four-tag detection, fluorescence detection involves optics and lasers that are relatively expensive and difficult to miniaturize. Electrochemical detection offers many advantages that might prove to be useful in the advancement of sequencing and DNA fragment analysis, but relatively little work has been done in this area. Several groups have investigated the use of redox active molecules for DNA hybridization studies. There are two commonly-used strategies utilized in detecting DNA hybridization with electrochemical detection. One strategy involves using diffusive electractive intercalators or metal catalysts to distinguish between single stranded and double stranded DNA on the surface of an electrode (Boon et al. (2000) Nature Biotechnology, 18: 1096-1100; Boon et al. (2000) Nature Biotechnology, 18: 1318; Welch and Thorp (1996) J. Phys. Chem., 100: 13829-13836; Mishima et al. (1997) Analytica Chimica Acta, 345: 45-50; Hashimoto et al. (1994) Anal. Chem. 66: 3830-3833; Pang and Abruna (1998) Anal. Chem., 70: 3162-3169; Hashimoto et al. (1994) Analytica Chimica Acta, 286: 219-224; Johnston et al. (1994) Inorganic Chemistry, 33: 6388-6390; Marrazza et al. (1999) Biosensors & Bioelectronics, 14: 43-51; Johnston et al. (1995) J. Amer. Chem. Soc., 117: 8933-8938; Welch et al. (1995) J. Phys. Chem., 99: 11757-11763; Millan and Mikkelsen (1993) Anal. Chem., 65: 2317-2323; Wang et al. (1997) Analytica Chimica Acta, 337: 41-48; Carter et al. (1989) Amer. Chem. Soc 11: 8901-8911; Georgopoulou et al. (2000) J. Chem. Society-Dalton Transactions, 2969-2974; Takenaka et al. (2000) Anal. Chem., 72: 1334-1341). The selectivity arises due to the increased affinity of the electroactive moiety for double stranded DNA, which increases the metal concentration at the electrode surface resulting in an increase in the electrochemical signal. For example, Thorp et al. have detected DNA hybridization through the catalytic oxidation of guanine by Ru(bpy)₃ at an indium tin oxide electrode (Napier et al. (1997) Bioconjugate Chemistry, 8: 906-913; Napier and Thorp (1997) Langmuir, 13: 6342-6344). An alternate strategy for electrochemical detection of DNA hybridization involves covalent modification of the DNA strand with a redox active molecule. A number of investigators have synthesized DNA that is modified with a ferrocene molecule covalently attached to the 5′ end (Ihara et al. (1996) Nucleic Acids Research, 24: 4273-4280; Ihara et al. (1997) Chemical Communications, 1609-1610; Mucic et al. (1996) Chemical Communications, 555-557; Xu et al. (2000) Analytica Chimica Acta, 411: 31-36; Takenaka et al. (1994) Anal. Biochem., 218: 436-443). Ihara used these modified oligonucleotides as the target strand in a hybridization assay. They used a 16-mer probe DNA strand modified with five phosphorothioate groups at the 5′ end that spontaneously attached to the gold sensing electrode. The modified gold electrode was then exposed to the target strand and hybridization was detected through the electrochemical response from the ferrocene tag. Grinstaff et al. (Hu et al. (2000) Inorganic Chemistry, 39: 2500-2504; Khan et al. (1999) Inorganic Chemistry, 38: 418-419,3A; Beilstein and Grinstaff (2000) Chemical Communications, 509-510; Tierney and Grinstaff (2000) Organic Letters, 2: 3413-3416; Tierney and Grinstaff (2000) J. Organic Chemistry, 65: 5355-5359), Yu et al. (2000) J. American Chemical Society, 122: 6767-6768, among others (Verheijen et al. (2000) Bioconjugate Chemistry, 11: 741-743; Price et al. (1996) Journal of the Chemical Society-Dalton Transactions, 4115-4120) have covalently incorporated a redox molecule into a phosphoramidite that is used during solid phase DNA synthesis. The tag is thus incorporated into the DNA strand at a predetermined point in the molecule and can be used in hybridization assays.

Previously, we have demonstrated the sensitivity and selectivity of sinusoidal voltammetry (SV) for the electrochemical detection of native DNA (Singhal et al. (1997) Anal. Chem., 69: 4828-4832; Singhal et al. (1997) Anal. Chem. 69: 3552-3557; Singhal et al. (1997) Anal. Chem., 69: 1662-1668; U.S. Pat. No. 5,958,215). In SV, the raw time domain data is converted into the frequency domain in order to: (1) better decouple faradaic signal from the background and (2) selectively identify molecules based on their unique frequency spectrum. For example, SV was used to detect native oligonucleotides at a copper microelectrode with detection limits in the low picomolar range (Singhal et al. (1997) Anal. Chem., 69: 4828-4832; Singhal et al. (1997) Anal. Chem. 69: 3552-3557).

### SUMMARY OF THE INVENTION

This invention provides a novel approach to the specific detection of redox-active moieties, e.g. in a population of redox-active moieties. In particular this invention provides a “phase-nulling” technique that can be used in the electrochemical detection of redox-active tags. The signal for each tag is selectively eliminated while the other tag's response remains virtually unchanged. This novel analysis scheme allows for the simple identification of a tag of interest in a complex matrix and is demonstrated with both flow injection analysis and capillary gel electrophoresis. The method is particularly well suited to nucleic acid sequencing applications.

Thus, in one embodiment, this invention provides a method of determining the sequence of a nucleic acid template. The method preferably involves i) generating and redox labeling sets of complementary sequencing fragments of the template where the sets of fragments terminating with the four different bases A, C, G, or T are each label labeled with a redox-active label that has an oxidation state distinct and distinguishable from the redox states of the labels labeling the other sets of fragments; ii) separating the sequencing fragments; iii) performing cyclic voltammetry on the sequencing fragments to produce a cyclic voltammogram for the redox-labeled sequencing fragments; iv) detecting the signal for each redox-active label at a phase angle out of phase with respect to the optimum phase angle for the redox-active label, where a drop-out of signal at said phase angle indicates the presence and/or amount of the redox-active label. In preferred embodiments, the dropout is as compared to the signal present at the phase common signal. In certain embodiments, the fragments are generated with a termination method employing primers, and terminators, and the primers or the terminators are labeled with the redox-active labels. Preferred terminators include dideoxy terminators (e.g. 2′,3′-dideoxyguanosine-5′-triphosphate, 7-deaza-2′,3′-dideoxyguanosine-5′-triphosphate, 2′,3′-dideoxyadenosine-5′-triphosphate, 2′,3′-dideoxythymidine-5′-triphosphate, and 2′,3′-dideoxycytidine-5′-triphosphate). In certain embodiments, the nucleoside triphosphates used for chain elongation are labeled with the redox-active labels. Preferred redox-active labels include, but are not limited to a porphyrin, an expanded porphyrin, a contracted porphyrin, a metallocene, a linear porphyrin polymer, and a porphyrin array. In certain embodiments, the redox-active labels comprise a ferrocene (e.g. alkyl ferrocene, ferrocene acetate, ferrocene carboxylate, alkyl ferrocene dimethylcarboxamide, acetyl ferrocene, propioly ferrocene, butyryl ferrocene, pentanoyl ferrocene, hexanoyl ferrocene, octanoyl ferrocene, benzoyl ferrocene, 1,1′-diacetyl ferrocene, 1,1′-dibutyryl ferrocene, 1,1′-dihexanoyl ferrocene, ethyl ferrocene, propyl ferrocene, n-butyl ferrocene, pentyl ferrocene, hexyl ferrocene, 1,1′-diethyl ferrocene, 1,1′-dipropyl ferrocene, 1,1′-dibutyl ferrocene, 1,1′-dihexyl ferrocene, cyclopentenyl ferrocene, cyclohexenyl ferrocene, 3-ferrocenoyl propionic acid, 4-ferrocenoyl butyric acid, 4-ferrocenylbutyric acid, 5-ferrocenylvaleric acid, 3-ferrocenoyl propionic acid esters, 4-ferrocenoyl butyric acid esters, 4-ferrocenyl butyric acid esters, 5-ferrocenylvaleric acid esters, dimethylaminomethyl ferrocene). In certain embodiments, the redox-active labels comprise a porphyrinic macrocycle substituted at a b-position or at a meso-position.

In certain embodiments, the voltammetry is performed at a single electrode. The voltammetry preferably utilizes a sinusoidal (or other periodic) waveform. In preferred embodiments, the cyclic voltammetry comprises converting voltammetric data into a time or frequency domain (e.g. via Fourier transform) to provide a frequency spectrum for a redox-active label. In certain embodiments, the cyclic voltammetry comprises selecting voltammetric data at a second, third, or higher harmonic frequency. The cyclic voltammetry preferably comprises selecting voltammetric data at a phase angle about 45 degrees to about 90 degrees, more preferably about 90 degrees out of phase with the optimum phase angle for the redox-active label whose presence is to be detected. The sequencing fragments are preferably separated (e.g. via chromatography, gel electrophoresis, capillary electrophoresis, and the like).

In another embodiment, this invention provides a chain-termination type nucleic acid sequencing method. The method preferably involves i) providing a template nucleic acid;

ii) annealing an oligonucleotide primer to a portion of the template nucleic acid thereby forming a primer-template hybrid; iii) adding a primer-extension reagent to the primer-template hybrid for extending the primer and forming a primer extension product, the primer extension reagent comprising nucleoside triphosphates; and iv) adding a terminator to the primer-template hybrid for causing specific termination of the primer extension and formation of a plurality of primer extension products where the terminator or the oligonucleotide primer is labeled with one of four redox-active tags where said redox-active tags have different and distinguishable oxidation states; v) separating said primer extension products; and vi) detecting the signal for each redox-active label at a phase angle out of phase with the optimum phase angle for the redox-active label, where a drop-out of signal at said that angle indicates the presence of the redox-active label.

This invention also provides a method of detecting a tagged analyte (e.g. a nucleic acid, a protein, an antibody, a sugar, a carbohydrate, a chain terminator, an analyte in an electrophoresis gel, a chromatographed analyte, etc.). The method involves i) providing at least two species of tagged analyte; ii) performing cyclic voltammetry on the tagged analyte to produce a cyclic voltammogram for the tagged analytes; iii) detecting the signal for a redox-active label at a phase angle out of phase with the optimum phase angle for the redox-active label that is to be detected, where a drop-out of signal at that phase angle indicates the presence of said redox-active label. In certain embodiments, the method comprises providing at least four species of tagged analyte where each species of tagged analyte is tagged with a redox-active label where the redox-active label attached to each species has an oxidation state different and distinguishable from the oxidation states of the redox-active labels attached to the other species of tagged analyte. Preferred redox-active labels include those redox-active labels described herein.

In certain embodiments, the voltammetry is performed at a single electrode. The voltammetry preferably utilizes a sinusoidal (or other periodic) waveform. In preferred embodiments, the cyclic voltammetry comprises converting voltammetric data into a time or frequency domain (e.g. via Fourier transform) to provide a frequency spectrum for a redox-active label. In certain embodiments, the cyclic voltammetry comprises selecting voltammetric data at a second, third, or higher harmonic frequency. The cyclic voltammetry preferably comprises selecting voltammetric data at a phase angle about 45 degrees to about 90 degrees, more preferably about 90 degrees out of phase with the optimum phase angle for the redox-active label whose presence is to be detected.

Also provided is a method of selective electrochemical detection of analytes in a complex mixture of analytes. The method involves i) labeling each analyte in the mixture with a redox label that generates an electrochemical signal that is different from the labels attached to other analytes in the mixture where said labeling provides labeled analytes; ii) performing cyclic voltammetry on the labeled analytes to produce a cyclic voltammogram for the labeled analytes; iii) detecting the signal for a redox-active label at a phase angle out of phase with the optimum phase angle for the redox-active label, where a drop-out of signal at said phase angle indicates the presence of the redox-active label.

In still yet another embodiment, this invention provides a computer-readable medium that can be used for directing an apparatus to detect and distinguish a plurality of redox-active tags where the redox-active tags have different and distinguishable oxidation states. The computer readable medium preferably comprises computer readable program code for directing a potentiostat in a cyclic voltammetric measurement to produce a cyclic voltammogram of the redox-active tags; and/or computer readable program code for detecting the signal for each redox-active label at a phase angle out of phase with the optimum phase angle for the redox-active label, where a drop-out of signal at the phase angle indicates the presence or amount of the redox-active label. In certain embodiments, the plurality of redox-active tags comprises four redox-active tags. In cetain embodiments, the code directs a cyclic voltammetric measurement (e.g. a sinusoidal voltammetric measurement) to be performed at a single electrode. The code for detecting the signal can comprise code for converting voltammetric data into a time or frequency domain to provide a frequency spectrum for a redox-active label. Such code can include a Fourier transform, fast Fourier transform, LaPlace transform, and the like. The code for detecting the signal can comprises code for selecting voltammetric data at a second, third, or higher harmonic frequency. The code for detecting the signal can comprise code for selecting voltammetric data at a phase angle about 45 degrees to about 90 degrees, preferably about 60 to about 90 degrees, more preferably about 80 to about 90 degrees, and most preferably about 90 degrees out of phase with the optimum phase angle for the redox-active label that is to be detected. Preferred computer readable media include, but are not limited to a magnetic disk, an optical disk, a chip, RAM, and ROM. In certain embodiments, the computer readable medium is supplied with or as a component of a nucleic acid sequencer.

Also provided is a computer-readable storage medium storing program code for causing a computer to detect and distinguish a plurality of redox-active tags where tej redox-active tags have different and distinguishable oxidation states, and the computer readable medium comprises program code directing a computer to detect the signal for each redox-active label at a phase angle out of phase with the optimum phase angle for that redox-active label, where a drop-out of signal at said phase angle indicates the presence and/or amount of the redox-active label. The computer readable storage medium of claim can further comprises program code for directing a potentiostat in a cyclic voltammetric measurement to produce a cyclic voltogram (e.g. a sinusoidal voltammogram) of the redox-active tags.

This invention also provides a kit for sequencing a nucleic acid. The kit preferably includes at least two, preferably at least three, and more preferably at least four redox-active tags where the redox active tags have different and distinguishable oxidation states. The redox-active tags can be provided isolated, attached to linkers, attached to elongation terminators, attached to primers, and the like. The kit can further comprise instructional materials teaching the detection of the signal for each redox-active label at a phase angle out of phase with the optimum phase angle for that redox-active label, where a drop-out of signal at said phase angle indicates the presence and/or amount of the redox-active label.

Certain kits for sequencing a nucleic acid comprise a plurality of redox-active tags where the redox active tags have different and distinguishable oxidation states; and a computer readable medium as described herein. The kit preferably includes at least two, preferably at least three, and more preferably at least four redox-active tags where the redox active tags have different and distinguishable oxidation states. The redox-active tags can be provided isolated, attached to linkers, attached to elongation terminators, attached to primers, and the like. The kit can further comprise instructional materials teaching the detection of the signal for each redox-active label at a phase angle out of phase with the optimum phase angle for that redox-active label, where a drop-out of signal at said phase angle indicates the presence and/or amount of the redox-active label.

In certain embodiments, this invention provides, in a computer system containing stored software programs, a method of detecting a tagged analyte from a plurality of tagged analytes, where the method comprises: ii) performing cyclic voltammetry on a plurality of tagged analytes where each species of tagged analyte is tagged with a redox-active label where the redox-active label attached to each species has an oxidation state different and distinguishable from the oxidation states of the redox-active labels attached to the other species of tagged analyte and the voltammetry produces a cyclic voltogram for the tagged analytes, where the cyclic voltammetry is performed by a potentiostat under control of said computer system; and ii) detecting the signal for a redox-active label at a phase angle out of phase with the optimum phase angle for that redox-active label, where a drop-out of signal at that phase angle indicates the presence of the redox-active label, where the detecting comprises analysis of said voltogram by said computer system.

This invention also provides a computer system, for detecting a redox active tag among a plurality of redox active tags. The computer system preferably comprises: a memory configured to store software programs; a data acquisition and control interface for acquiring data from a potentiostat; and a computer readable medium comprising computer readable program code for directing the potentiostat in a cyclic voltammetric measurement to produce a cyclic voltammogram of the redox-active tags; and/or computer readable program code for detecting the signal for each redox-active label at a phase angle out of phase with the optimum phase angle for the redox-active label that is to be detected, where a drop-out of signal at that phase angle indicates the presence and/or amount of that redox-active label.

Definitions

The term “oxidation” refers to the loss of one or more electrons in an element, compound, or chemical substituent/subunit. In an oxidation reaction, electrons are typically lost by atoms of the element(s) involved in the reaction. The charge on these atoms then becomes more positive. The electrons are lost from the species undergoing oxidation and so electrons appear as products in an oxidation reaction. An oxidation is taking place in the reaction Fe^{2 +}(aq)--≦Fe³⁺(aq)+e⁻ because electrons are lost from the species being oxidized, Fe²⁺(aq), despite the apparent production of electrons as “free” entities in oxidation reactions. Conversely the term reduction refers to the gain of one or more electrons by an element, compound, or chemical substituent/subunit.

An “oxidation state” refers to the electrically neutral state or to the state produced by the gain or loss of electrons to an element, compound, or chemical substituent/subunit. In a preferred embodiment, the term “oxidation state” refers to states including the neutral state and any state other than a neutral state caused by the gain or loss of electrons (reduction or oxidation).

The terms “different and distinguishable” when referring to two or more oxidation states means that the net charge on the entity (atom, molecule, aggregate, subunit, etc.) can exist in two different states. The states are said to be “distinguishable” when the difference between the states is greater than thermal energy at room temperature (e.g. 0° C. to about 40° C.).

The term “electrode” refers to any medium capable of transporting charge (e.g. electrons) to and/or from a redox-active species. Preferred electrodes are metals or conductive organic molecules. The electrodes can be manufactured to virtually any 2-dimensional or 3-dimensional shape (e.g. discrete lines, pads, planes, spheres, cylinders, etc.).

A “redox-active” compound, or molecule refers to a compound or molecule capable of being oxidized or reduced. A redox-active tag is a redox-active compound or molecule that can be or is attached to a moiety that is to be detected. The redox-active tag provides a detectable signal or property (e.g. oxidation state) that provides an indication of the presence and/or amount of a moiety tagged with such a tag.

The term “E_{1/2}” refers to the practical definition of the formal potential (E^{o}) of a redox process as defined by E=E^{o}+(RT/nF)ln(Dₒₓ/D_{red}) where R is the gas constant, T is temperature in K (Kelvin), n is the number of electrons involved in the process, F is the Faraday constant (96,485 Coulomb/mole), Dₒₓ is the diffusion coefficient of the oxidized species and D_{red} is the diffusion coefficient of the reduced species.

The term “optimum phase angle” for a redox active species (tag) refers to the phase angle of an electrochemical measurement in the frequency domain (e.g. a cyclic voltammetric measurement) that gives maximum current for the signal (i.e. greatest S/N ratio).

The phrase “drop out of signal at a phase angle” indicates a dimunition or elimination of a signal at a particular phase angle as compared to that signal at a different phase angle. The dimumition can be any detectable dimunition, preferably a dimunition of at least 5%, preferably of at least 10%, more preferably of at least 15% or 20%, most preferably of at least 30%, at least 50%, or at least 80%. In certain embodiments, the dimunition is a statistically significant dimunition (e.g. at the 10% confidence level, more preferably at the 5% confidence level and most preferably at the 1% confidence level). In certain embodiments, the dimunition is relative to the signal at the optimum phase angle for that redox-active species. Where a collection of redox-active species is present, in particularly preferred embodiments, the dimunition is as compared to the signal at the phase common response.

A “voltage source” is any source (e.g. molecule, device, circuit, etc.) capable of applying a voltage to a target (e.g. an electrode).

A “voltammetric device” is a device capable of measuring the current produced in an electrochemical cell as a result of the application of a voltage or change in voltage.

An “amperometric device” is a device capable of measuring the current produced in an electrochemical cell as a result of the application of a specific potential field (“voltage”).

A “potentiometric device” is a device capable of measuring potential across an interface that results from a difference in the equilibrium concentrations of redox molecules in an electrochemical cell.

A “voltammogram” refers to the data set produced by a voltammetric measurement (e.g. a cyclic voltammogram is the data set produced by a cyclic voltammetric measurement). The data set can be permanently or transiently displayed in electronic or other forms. In certain embodiments, the voltammogram need not be display, but can simply exist, e.g. as a data set on a computer readable medium (e.g. dynamic memory, static memory, optical storage, magnetic storage, and the like) and be accessed for subsequent processing. The voltammogram can be the raw data from the measurement of a transform of such raw data (e.g. background subtracted, and/or Fourier transformed, and the like).

A “coulometric device” is a device capable of the net charge produced during the application of a potential field (“voltage”) to a redox-active species.

A “cyclic voltammeter” is a voltammetric device capable of determining the time and/or frequency domain properties of a redox-active species (i.e. a device capable of performing cyclic voltammetry).

Cyclic voltammetry, as used herein, refers to voltammetry using a periodic waveform (e.g. sine, cosine, triangle, or any combination thereof) as an excitation potential. Although often linear, such waveforms need not be so limited.

The term “sinusoidal voltammetry” refers to cyclic voltametry using a periodic “excitation voltage” that is sinusoidal.

The term “porphyrinic macrocycle” refers to a porphyrin or porphyrin derivative. Such derivatives include porphyrins with extra rings ortho-fused, or ortho-perifused, to the porphyrin nucleus, porphyrins having a replacement of one or more carbon atoms of the porphyrin ring by an atom of another element (skeletal replacement), derivatives having a replacement of a nitrogen atom of the porphyrin ring by an atom of another element (skeletal replacement of nitrogen), derivatives having substituents other than hydrogen located at the peripheral (meso-, β-) or core atoms of the porphyrin, derivatives with saturation of one or more bonds of the porphyrin (hydroporphyrins, e.g., chlorins, bacteriochlorins, isobacteriochlorins, decahydroporphyrins, corphins, pyrrocorphins, etc.), derivatives obtained by coordination of one or more metals to one or more porphyrin atoms (metalloporphyrins), derivatives having one or more atoms, including pyrrolic and pyrromethenyl units, inserted in the porphyrin ring (expanded porphyrins), derivatives having one or more groups removed from the porphyrin ring (contracted porphyrins, e.g., corrin, corrole) and combinations of the foregoing derivatives (e.g. phthalocyanines, sub-phthalocyanines, and porphyrin isomers). Preferred porphyrinic macrocycles comprise at least one 5-membered ring. A number of porphyrinic macrocycles are described in WO 01/03126.

The term “ferrocene” includes ferrocene and ferrocene derivatives, e.g. alkyl ferrocene, ferrocene acetate, ferrocene carboxylate, alkyl ferrocene dimethylcarboxamide, acetyl ferrocene, propioly ferrocene, butyryl ferrocene, pentanoyl ferrocene, hexanoyl ferrocene, octanoyl ferrocene, benzoyl ferrocene, 1,1′-diacetyl ferrocene, 1,1′-dibutyryl ferrocene, 1,1′-dihexanoyl ferrocene, ethyl ferrocene, propyl ferrocene, n-butyl ferrocene, pentyl ferrocene, hexyl ferrocene, 1,1′-diethyl ferrocene, 1,1′-dipropyl ferrocene, 1,1′-dibutyl ferrocene, 1,1′-dihexyl ferrocene, cyclopentenyl ferrocene, cyclohexenyl ferrocene, 3-ferrocenoyl propionic acid, 4-ferrocenoyl butyric acid, 4-ferrocenylbutyric acid, 5-ferrocenylvaleric acid, 3-ferrocenoyl propionic acid esters, 4-ferrocenoyl butyric acid esters, 4-ferrocenyl butyric acid esters, 5-ferrocenylvaleric acid esters, dimethylaminomethyl ferrocene, and the like.

The term “porphyrin” refers to a cyclic structure typically composed of four pyrrole rings together with four nitrogen atoms and two replaceable hydrogens for which various metal atoms can readily be substituted. A typical porphyrin is heme.

The term “working electrode” typically used to refer to one or more electrodes that are used to read the oxidation state of a redox-active species.

The term “reference electrode” is typically used to refer to one or more electrodes that provide a reference (e.g. a particular reference voltage) for measurements recorded from the working electrode. In certain embodiments, the excitation waveform is applied at the reference electrode.

The term “oligonucleotide primer” refers to an oligonucleotide or polynucleotide that, when annealed to a template nucleic acid, is capable of being extended from a 3′-end in the presence of primer extension reagents. Typically, an oligonucleotide primer will include a hydroxyl group at the 3′-position of a 3′-terminal nucleotide.

The term “phosphate analog” refers to analogs of phosphate wherein the phosphorous atom is in the +5 oxidation state and one or more of the oxygen atoms is with a non-oxygen moiety, exemplary analogs including phosphorothioate, phosphorodhioate, phosphoroselenoate, phosphorodiselenoate, phosphoroanilothioate, phosphoranilidate, phosphoramidate, boronophosphates, and the like, including associated counterions, e.g., H, NH₄, Na, and the like if such counterions are present.

As used herein the term “primer-extension reagent” means a reagent including components necessary to effect the enzymatic template-mediated extension of a nucleic acid (e.g. an oligonucleotide) primer. Preferably, primer extension reagents include: (i) a polymerase enzyme, e.g., a thermostable polymerase enzyme such as Taq polymerase; (ii) a buffer; and (iii) 2′-deoxynucleotide triphosphates, e.g., 2′-deoxyuridine-5′-triphosphate, 2′-deoxyguanosine-5′-triphosphate, 2′-deoxy-7-deazadeoxyguanosine-5′-triphosphate, 2′-deoxyadenosine-5′-triphosphate, 2′-deoxythymidine-5′-triphosphate, 2′-deoxycytidine-5′-triphosphate.

As used herein, the term “terminator” refers to a species that when incorporated into a primer extension product blocks further elongation of the product. Exemplary terminators include 2′,3′-dideoxynucleotides, e.g., 2′,3′-dideoxyguanosine-5′-triphosphate, 7-deaza-2′,3′-dideoxyguanosine-5′-triphosphate, 2′,3′-dideoxyadenosine-5′-triphosphate, 2′,3′-dideoxythymidine-5′-triphosphate, and 2′,3′-dideoxycytidine-5′-triphosphate.

As used herein, the term “template nucleic acid” refers to any nucleic acid which can be presented in a single stranded form and is capable of annealing with a nucleic acid primer. Exemplary template nucleic acids include DNA, RNA, which DNA or RNA can be single stranded or double stranded. More particularly, template nucleic acid can be genomic DNA, messenger RNA, cDNA, DNA amplification products from a PCR reaction, and the like. Methods for preparation of template DNA may be found elsewhere (ABI PRISM.TM. Dye Primer Cycle Sequencing Core Kit Protocol).

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The terms “nucleic acid”, or “oligonucleotide” or grammatical equivalents herein refer to at least two nucleotides covalently linked together. Nucleic acids of the present invention are single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, 0-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Letsinger et al. (1988) J. Am. Chem. Soc. 110:4470; Letsinger et al. (1994) Nucleoside & Nucleotide 13:1597; Chapters 2 and 3, ACS Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ACS Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

### BRIEF DESCRIPTION OF THE DRAWINGS

FIG. 1 illustrates electrochemically-tagged oligonucleotides. The base sequence for all the probes is a 20-mer oligonucleotide, the T3 primer (5′ AAT TAA CCC TCA CTA AAG GG 3′, SEQ ID NO: 1). Each redox active tag is attached to the 5′ adenosine moiety, A. AF. Alkyl ferrocene-tagged T3 primer FA. Ferrocene acetate-tagged T3 primer FC. Ferrocene carboxylate-tagged T3 primer AFD. Alkyl ferrocene dimethylcarboxamide-tagged T3 primer.

FIGS. 2A through 2D illustrate cyclic voltammetry of the four tagged t3 primers. Cyclic Voltammetry was performed in static solution from −200 to 650 mV vs. Ag/AgCl at 1 V/s as described herein. The CVs (not background-subtracted) for 100 μM solutions of: AF T3 primer (FIG. 2A), FA T3 primer (FIG. 2B), FC T3 primer (FIG. 2C), and AFD T3 primer (FIG. 2D) are shown.

FIG. 3 illustrates background-subtracted sinusoidal voltammetric frequency spectra for the four electrochemically-tagged T3 primers. Sinusoidal voltammetry of the four ferrocene-tagged T3 primers was performed by applying an 11 Hz sine wave, -200 to 650 mV vs. Ag/AgCl (sat'd KCl). The running electrolyte was Genetic Analyzer buffer (lx). Each analyte plug was injected sequentially into the capillary FIA system. The background subtracted current response from the height of each signal was used to create each frequency domain response. The information shown in the frequency domain is plotted as: frequency (Hz) (x-axis), current magnitude (nA) (z-axis), and phase angle (degrees) (y-axis). The background-subtracted SV frequency spectra of sequential 10 μM FIA injections of AF T3 primer (A), FA T3 primer (B), FC T3 primer (C), and AFD T3 primer (D) are shown.

FIG. 4, panels A through 4E illustrate a scheme for selective elimination of the signal for individually tagged T3 primers in time course data. The time course information shown here represents the same data set as in FIG. 3. Panel A: Phase-Common response: the time domain signal at the third harmonic (33 Hz) was selected to show all of the four tagged T3 primers locked at a common phase angle of 160 degrees. (panels B-E). Phase-Nulled response for each tagged T3 primer: Panel B: AF tag—The time domain response is locked at 168 degrees, which is exactly +90 degrees out of phase with the optimum phase angle for the AF tag. Panel C: FA tag—The time domain response is locked at 139 degrees, which is +90 degrees out of phase with the FA tag. Panel D: FC tag—The time domain response is locked at 83 degrees, which is −90 degrees out of phase with the FC tag. Panel E: AFD tag—The time domain response is locked at 41 degrees, which is −90 degrees out of phase with the AFD tag.

FIG. 5 illustrates background-subtracted sinusoidal voltammetry (SV) frequency spectra obtained under capillary gel electrophoresis (CGE) conditions. This figure illustrates the background-subtracted SV frequency spectrum obtained for a 100 nM electrophoretic injection of the FA tag (●) and the background-subtracted SV frequency spectrum obtained for a 100 nM electrophoretic injection of the FC tag (▪). An 11-Hz sine wave, -200 to 800 mV vs. Ag/AgCl (sat'd KCl), was used as the excitation. The CGE conditions were as follows; 20 μm i.d. capillary, 19 cm to the detector, and a run voltage of −305 V/cm. The FC tag was injected for 12 seconds at −174 V/cm. Immediately after, the FA tag was injected for 12 seconds at −174 V/cm. The electrophoresis buffer was replaced and a field of −305 V/cm was applied continuously to elute the tags from the CGE column past the electrochemical detector. The electrochemical conditions were as follows: an 11 Hz sine wave, -200 to 800 mV vs Ag/AgCl (sat'd KCl).

FIGS. 6A through 6C illustrate the selective elimination of the signal for individually tagged t3 primers in capillary gel electrophoresis. FIG. 6A: Phase-Common Signal: The time domain at the third harmonic (33 Hz) for the FC and FA T3 primers locked at a common phase angle of 36 degrees. Phase-nulled signal: (FIG. 6B). The CGE time course showing the elution of the two tags locked at 123 degrees, which is exactly +90 degrees out of phase with the FC T3 primer signal. FIG. 6C: The CGE time course showing the elution of the two tags locked at 161 degrees, which is exactly +90 degrees out of phase with the FA T3 primer signal. The time domain data is from the same CGE/SV experiment in FIG. 5.

FIG. 7 is a block diagram of an exemplary computer system upon which the present invention can be implemented.

### DETAILED DESCRIPTION

This invention pertains to a novel approach to the use of redox-active labels and to detecting and discriminating redox-active labels in a complex mixture of such labels. In preferred embodiments, this invention provides a novel “calling” scheme that utilizes the unique frequency responses of the redox-active (electrochemical) tags. All the data can be collected at a single electrode and each tag can be selected and identified through software processing of a single data set.

The tags and analytic methods of this invention can be applied to detect and/or quantitate one or more species in essentially any collection of moieties that are to be labeled (e.g. antibodies, proteins, lipids, nucleic acids, sugars, carbohydrates, inorganic molecules, objects of manufacture, various beads and other nanoparticles, and the like). In a particularly preferred embodiment, the tags and methods of this invention are particularly useful in nucleic acid sequencing applications, or various high-throughput screening applications where it is desirable to screen for and detect each tagged analyte in a plurality (mixture) of analytes (e.g. in a lane of an electrophoresis gel, in a capillary electrophoresis tube, in a well in a microtiter plate, and the like).

In particularly preferred embodiments, a novel “tag-calling” scheme is presented utilizing the unique frequency responses for the electrochemical tags, where all data is obtained at a single electrode placed at the end of a flow stream, and each tag can be selected and identified through software processing of a single data set. Each tag has a unique SV frequency spectrum that can be easily identified, e.g., in the frequency domain. In preferred embodiments, the discrimination of one tag versus all others is accomplished through a “phase-nulling” technique. In this approach, the signal for each tag is selectively eliminated while the other three responses remain virtually unchanged.

In the case of oligonucleotides, this analysis scheme allows for the selective identification of each tagged oligonucleotide eluting in sieving polymer capillary gel electrophoresis with a separation efficiency of 2×10⁶ theoretical plates per meter or better. This separation efficiency is sufficient to perform nucleic acid sequencing.

I. Detection and Discrimination of Redox-Active Tags.

In certain embodiments, this invention provides a novel approach to the detection and discrimination of one or more redox-active tags, particularly in a population of such tags. In preferred embodiments, the redox-active tags are used to provide a detectable property of signal indicating the presence and/or concentration of one or more target analytes. In particularly preferred embodiments, a plurality of redox-active tags that have different and distinguishable oxidation states are used to label each moiety that it is desired to detect. Typically, a different species of redox-active tag is used for each species of analyte that it is desired to detect.

The redox-active tags can be detected using any of a wide variety of electrochemical technologies including amperometric methods (e.g. chronoamperometry), coulometric methods (e.g. chronocoulometry), voltammetric methods (e.g., linear sweep voltammetry, cyclic voltammetry, pulse voltammetries, sinusoidal voltammetry, etc.), any of a variety of impedance and/or capacitance measurements, and the like. Such readouts can be performed in the time and/or frequency domain.

In preferred embodiments, cyclic voltammetric methods are used. Data acquisition in sinusoidal voltammetry is preferably performed as described by Brazill et al. (2000) Anal. Chem. 72: 5542-5548. Briefly, a computer-generated time-varying potential (e.g. a triangle wave, a sine wave, etc) scans through the potential window of interest. The data is acquired using standard methods, e.g. a data acquisition system, preferably at a frequency substantially higher (e.g. preferably 50 fold higher or greater, more preferably 100 fold or greater, and most preferably 150- or 200-fold or greater), than the scan (excitation) frequency.

In particularly preferred embodiments, frequency domain information is obtained by continuous conversion of each scan, e.g., via the application of a Fast Fourier Transform (FFT). Other approaches can also be used to extract frequency domain information. Such approaches include, but are not limited to LaPlace transform, wavelet analysis, Wigner distriubition, and the like. This frequency domain data provides voltammetric information characteristic of each redox active species (the SV frequency spectrum) and time course information (current versus time), as described previously.

The frequency spectrum for a specific analyte consists of a series of vectors (represented as magnitude and phase) at each of a number of harmonics (e.g. at least 2, preferably 5, more preferably 10 or more). To isolate the information particular to a given analyte, a background frequency spectrum is subtracted from the entire data set. While definition of the background spectrum is somewhat arbitrary, a convenient measure is at least one scan, preferably the average of the two, more preferably first five, and most preferably the first scans, that typically represent the background processes (capacitive and faradaic) at the electrode surface (in the absence of analyte). It will be appreciated that the background scan can be varied to optimize signal to noise ratio or other parameters.

The frequency spectrum for the analyte of interest is simply defined as the background-subtracted current vector (magnitude and phase) at the highest part of the signal.

In addition to the SV frequency spectrum, time course data can be obtained at each harmonic frequency element by performing the “digital equivalent of a lock-in amplifier”. Analogous to an analog lock-in amplifier, the instantaneous current is monitored at the optimum phase angle for the signal of interest, thus increasing the sensitivity and selectivity over traditional voltammetric techniques. Maximum sensitivity is achieved if the background phase angle is ±90 degrees out of phase with the optimum phase angle for the signal. Therefore, the analyte is typically monitored at the phase angle that gives the maximum current for the signal and minimum background, which increases the S/N for the measurement. Finally, the phase-optimized time course data can, optionally, be digitally filtered, e.g., with a low-pass filter using a boxcar averaging routine or other routine.

This “digital lock-in approach” can also be used to distinguish between different molecules in the time domain. Selective discrimination between analytes with different electrochemical characteristics (formal potential, kinetics, etc.) is accomplished by identifying the frequency component where signal due to the analyte is closest to 90° out of phase with all other components. Using such a “phase nulling approach” with careful selection of the frequency and phase angle one can isolate the signal of the component of interest in almost any complex matrix.

In preferred embodiments, a frequency and phase angle are determined where all components (tag signals) can be monitored with similar sensitivity (i.e. monitored at a single frequency and phase angle where each tag is equally represented). This “phase-common” signal can be obtained at any given frequency, simply by examining the difference between signals as a function of phase angle. The variance between each pair of components is calculated at each phase angle (e.g., i_{ab}=(iₐ−i_{b})²), and the overall variance (i_{TOTAL}=Σiᵢ/n, where iᵢ are the individual variances and n is the total number of combinations), representing the sum of all the individual variances, is calculated and minimized to find the optimum phase angle for all components. This allows rapid identification of a single phase angle that can be used to monitor all the subject tags with only slight variation from their maximum current response.

At the frequency and phase angle producing the “phase-common” signal, a large signal is observed for all four tags, with typically minimal loss in signal compared to that observed at each of the optimum phase angles. The phase-nulled signal for each redox-active tag can then be obtained at the same frequency (e.g., third harmonic) by locking-in at a phase angle that is out of phase from the optimum for each tag. The phase angle is preferably either +or −90 degrees out of phase. The signal corresponding to the phase-nulled component is effectively diminished while the other three tags are relatively unaffected. When compared to the phase-common signal, it is easy to determine which peak has been removed. This method thus allows rapid “tag calling” on a single set of data simply by identifying the peaks that disappear.

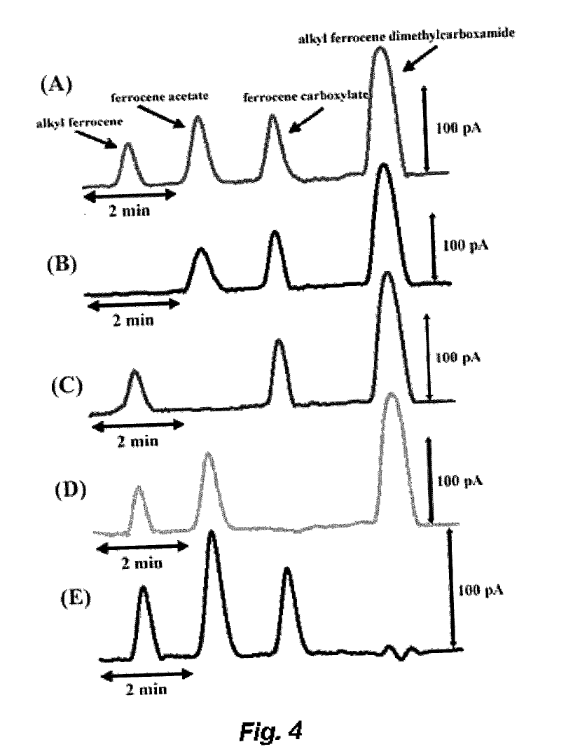

This tag-calling approach is illustrated in FIG. 4 which shows a scheme for selective elimination of the signal for individually tagged T3 primers in time course data. FIG. 4A shows the phase common response. The time domain signal at the third harmonic (33 Hz) was selected to show all of the four tagged T3 primers locked at a common phase angle of 160 degrees. FIGS. 4B through 4E show the phase-nulled response for each tagged T3 primer: Thus, for example, FIG. 4B illustrates nulling of the AF tag response. The time domain response is locked at 168 degrees, which is exactly +90 degrees out of phase with the optimum phase angle for the AF tag. Similarly, FIGS. 4C, 4D, and 4E illustrate nulling of the FA tag, the FC tag, and the AFD tag respectively.

Such selective nulling of each redox-active tag can be constantly performed over the course of a measurement. Thus, for example, where the output of a capillary electrophoresis experiment (e.g. nucleic acid sequencing reaction) is being assayed, a detection electrode is positioned at the end of the capillary tube or at one or more discrete points along the tube. The above-described phase nulling is performed for each tag expected to be present in the experiment. Dropout of the signal for the tag indicates the presence of that tag at that time.

A wide variety selection of equipment for such electrochemical measurement is commercially available (see, e.g., Sycopel, Inc.; Solartron Analytic potentiostat; Princeton Applied Research, Pinnacle Technology, Inc. Multichannel Potentiostats, etc.). Commercial data acquisition systems often have integrated and/or associated data acquisitions systems and appropriate software that allows the programming of data analysis functions.

II. Redox-Active Tags.

A wide variety of molecules can be used as redox-active tags according to this invention. Preferred molecules include, but are not limited to a porphyrinic macrocycle, a metallocene, a linear polyene, a cyclic polyene, a heteroatom-substituted linear polyene, a heteroatom-substituted cyclic polyene, a tetrathiafulvalene, a tetraselenafulvalene, a metal coordination complex, a buckyball, a triarylamine, a 1,4-phenylenediamine, a xanthene, a flavin, a phenazine, a phenothiazine, an acridine, a quinoline, a 2,2′-bipyridyl, a 4,4′-bipyridyl, a tetrathiotetracene, and a peri-bridged naphthalene dichalcogenide. Even more preferred molecules include a porphyrin, an expanded porphyrin, a contracted porphyrin, a metallocene (e.g. a ferrocene), a linear porphyrin polymer, and a porphyrin array. Certain particularly preferred redox-active tags include a porphyrinic macrocycle substituted at a β-position or at a meso-position (e.g. as described in WO 01/03126).

Particularly preferred redox-active tags include metallocenes, more preferably ferrocenes having different substitutents attached to the ferrocene ring, where the electron donating or withdrawing character of the substituent alters the half-wave potential of the modified metallocene. Particularly preferred ferrocenes include, but are not limited to alkyl ferrocene, ferrocene acetate, ferrocene carboxylate, alkyl ferrocene dimethylcarboxamide, acetyl ferrocene, propioly ferrocene, butyryl ferrocene, pentanoyl ferrocene, hexanoyl ferrocene, octanoyl ferrocene, benzoyl ferrocene, 1,1′diacetyl ferrocene, 1,1′-dibutyryl ferrocene, 1,1′-dihexanoyl ferrocene, ethyl ferrocene, propyl ferrocene, n-butyl ferrocene, pentyl ferrocene, hexyl ferrocene, 1,1′-diethyl ferrocene, 1,1′-dipropyl ferrocene, 1,1′-dibutyl ferrocene, 1,1′-dihexyl ferrocene, cyclopentenyl ferrocene, cyclohexenyl ferrocene, 3-ferrocenoyl propionic acid, 4-ferrocenoyl butyric acid, 4-ferrocenylbutyric acid, 5-ferrocenylvaleric acid, 3-ferrocenoyl propionic acid esters, 4-ferrocenoyl butyric acid esters, 4-ferrocenyl butyric acid esters, 5-ferrocenylvaleric acid esters, dimethylaminomethyl ferrocene, and the like.

As indicated above, the redox-active tags are selected so that each tag that is to be present, e.g. in a mixture of tags, has a different and distinguishable oxidation state from the other tags in that mixture.

Control over the oxidation state(s) of the redox-active tags of this invention can be regulated through through synthetic design. The oxidation (redox) potential can be tuned with precision by choice of base molecule(s), associated metals and peripheral substituents (Yang et al. (1999) J. Porphyrins Phthalocyanines, 3: 117-147).

For example, in the case of porphyrins, Mg porphyrins are more easily oxidized than Zn porphyrins, and electron withdrawing or electron releasing aryl groups can modulate the oxidation properties in predictable ways. The effects of metals on metalloprophyrin oxidation potentials are well known (Fuhrhop and Mauzerall (1969) J. Am. Chem. Soc., 91: 4174-4181) and provide a strong foundation for designing redox-active tags with oxidation states.

The design of compounds with predicted redox potentials is well known to those of ordinary skill in the art. In general, the oxidation potentials of redox-active units or subunits are well known to those of skill in the art and can be looked up (see, e.g., Handbook of Electrochemistry of the Elements). Moreover, in general, the effects of various substituents on the redox potentials of a molecule are generally additive. Thus, a theoretical oxidation potential can be readily predicted for any potential data storage molecule. The actual oxidation potential, particularly the oxidation potential of the information storage molecule(s) or the information storage medium can be measured according to standard methods. Typically the oxidation potential is predicted by comparison of the experimentally determined oxidation potential of a base molecule and that of a base molecule bearing one substituent in order to determine the shift in potential due to that particular substituent. The sum of such substituent-dependent potential shifts for the respective substituents then gives the predicted oxidation potential.

The redox-active tags can be prepared according to routine methods well known to those of skill in the art, see, e.g., Prathapan et al. (1993) J. Am. Chem. Soc., 115: 7519-7520, Wagner et al. (1995) J. Org. Chem., 60: 5266-5273, Nishino et al. (1996) J. Org. Chem., 61: 7534-7544, Wagner et al. (1996) J. Am. Chem. Soc., 118: 11166-11180, Strachan et al. (1997) J. Am. Chem. Soc., 119: 11191-11201, and Li et al. (1997) J. Mater. Chem., 7: 1245-1262. These papers describe various strategies for the synthesis of a number of multi-porphyrin (porphyrinic macrocycle) compounds.

In certain preferred embodiments, the synthesis of phorphyrinic macrocylce tags involves a room temperature one-flask synthesis of meso-substituted porphyrins (Lindsey et al. (1987) J. Org. Chem. 52: 827-836, Lindsey et al. (1994) J. Org. Chem. 59: 579-587, Li et al. (1997) Tetrahedron, 53: 12339-12360.), and/or incorporation of bulky groups around the porphyrin to achieve enhanced solubility in organic solvents (Lindsey and Wagner (1989) J. Org. Chem., 54: 828-836), and or a one-flask synthesis of dipyrromethanes, key building blocks in the synthesis of porphyrins bearing 2-4 different meso-substituents (Lee and Lindsey (1994) Tetrahedron, 50: 11427-11440, Littler et al. (1999) J. Org. Chem. 64: 1391-1396). Certain syntheses involve a of trans-substituted porphyrins without acidolytic scrambling (Littler et al. (1999) J. Org. Chem. 64: 2864-2872). In addition it is noted that the rational synthesis of porphyrins bearing up to 4 different meso-substituents is described by Lee et al. (1995) Tetrahedron, 51: 11645-11672, Cho et al. (1999) J. Org. Chem. 64: 7890-7901, while mild methods for inserting magnesium (Lindsey and Woodford (1995) Inorg. Chem. 34: 1063-1069, O'Shea et al. (1996) Inorg. Chem., 35: 7325-7338) or other metals (Buchler, J. W. In The Porphyrins; Dolphin, D. Ed.; Academic Press: New York. 1978; Vol. 1, pp. 389-483) into porphyrins are also known.

In one embodiment, building blocks are synthesized using methods described by Wagner et al. (1996) J. Am. Chem. Soc., 118:11166-11180, Strachan et al. (1997) J. Am. Chem. Soc., 119: 11191-11201, Wagner et al. (1996) J. Am. Chem. Soc., 118: 3996-3997, Li et al. (1997) J. Mater. Chem., 7: 1245-1262; Lindsey et al. (1994) Tetrahedron, 50: 8941-8968; Wagner et al. (1994) J. Am. Chem. Soc., 116: 9759-9760; Lindsey and Wagner (1989) J. Org. Chem., 54: 828-836; Lee and Lindsey (1994) Tetrahedron, 50: 11427-11440; Lee et al. (1995) Tetrahedron, 51: 11645-11672; Lindsey and Woodford (1995) Inorg. Chem. 34: 1063-1069; and Wagner et al. (1995) J. Org. Chem., 60: 5266-5273.

In particularly preferred embodiments, metallocene redox-active tags (e.g. ferrocene tags) of this invention can be synthesized according to the procedure of Ihara et al. (see, e.g., Ihara et al. (1996) Nucleic Acids Research, 24: 4273-4280; Ihara et al. (1997) Chemical Communications, 1609-1610). Briefly, an activated N-hydroxysuccinimide (NHS) ester of ferrocene carboxylic acid and ferrocene acetic acid is synthesized using NHS and dicyclohexylcarbodiimide. The product can be purified on a column of silica gel (Merck 60, methylene chloride eluent). The activated ester (dissolved in dimethyl sulfoxide) can then be linked to an oligonucleotide, e.g., a 5′ amine terminated T3 primer in a 0.5 M NaHCO₃/Na₂CO₃ (pH=9) buffer solution overnight. The reaction mixture can then be diluted with water and chromatographed on a NAP-10 column (Pharmacia Sephadex G-25). The absorbance was measured at 260 nm and used to determine which fractions contained the nucleicacid. Those fractions are combined, lyophilized, and purified by RP-HPLC.

It is also noted that ferrocene syntheses are described in WO 01/06016 and by Blom et al. (1987) Transition Met. Chem., 12: 301-306. In addition, dedideoxy ferrocene synthesis is described by Blanc et al. (2001) J. Bioconjugate Chem., 12, (2001) 396-405.

Using the teachings provided in the references cited herein and illustrated in the Examples, one of skill can routinely synthesize a wide number of different and distinguishable redox-active tags.

II. Use of Tagged Moieties for Detection of Analytes.

The redox-active tags and detection methods of this invention can be used to detect essentially any moiety that it is desired to detect. Such moieties include, but are not limited to antibodies, proteins, lipids, nucleic acids, sugars, carbohydrates, inorganic molecules, objects of manufacture, various beads (e.g. quartz or other mineral, glass, plastic, other resin) and other nanoparticles, and the like.

Where the redox-active tag is attached to the target analyte, detection of the tag provides a direct indication of the presence of the target analyte (e.g., a nucleic acid fragment).

It will be appreciated, however, that it is not necessary to directly label the analyte that is to be detected with the redox-active tag. In certain assays, e.g. competitive assays ligands that compete with the analyte for a binding site, e.g. for an antibody binding site can be labeled with the redox-active tags. Detection of the labeled ligand (either the ligand bound to the binding site or the free ligand competed away from the binding site by the target analyte) provides a measure of the presence and/or amount of target analyte.

In certain “sandwich” assays the target analyte is bound by a ligand or an antibody that provides a domain that itself can be bound by a second ligand or antibody where the second ligand or antibody is labeled with the redox-active tag to indicate the presence of the target species corresponding to that tag.

The redox-active tags can be attached to the target analyte(s) or other moieties according to standard methods well known to those of skill in the art. Means of coupling reactive moieties such as redox-active tags to a target are well known to those of skill in the art. Linkage of the redox-active tag to a moiety can be covalent, or by charge or other non-covalent interactions. The target moiety and/or the redox-active tag can be specifically derivatized to provide convenient linking groups (e.g. hydroxyl, amino, sulfhydryl, etc.). Covalent linkage of the redox-active tag to the target analyte can be direct or through a covalent linker. Where the moiety to be labeled is a protein, antibody, etc., it is noted that proteins contain a variety of functional groups; e.g., carboxylic acid (COOH) or free amine (—NH2) groups, which are available for reaction with a suitable functional group on either the redox-active tag or on a linker attached to the surface. Similarly nucleic acids bear free hydroxyl groups suitable for coupling tags through the sugar moiety. In certain embodiments, the tag can be coupled to the nucleic acid through the base.

Generally linkers are either hetero- or homo-bifunctional molecules that contain two or more reactive sites that may each form a covalent bond with the respective binding partner (i.e. surface or ecotin variant). Linkers suitable for joining biological binding partners are well known to those of skill in the art. For example, a protein molecule may be linked by any of a variety of linkers including, but not limited to a peptide linker, a straight or branched chain carbon chain linker, or by a heterocyclic carbon linker. Heterobifunctional cross linking reagents such as active esters of N-ethylmaleimide have been widely used. See, for example, Lemer et al. (1981) Proc. Nat. Acad. Sci. (USA), 78: 3403-3407 and Kitagawa et al. (1976) J. Biochem., 79: 233-236, and Birch and Lennox (1995) Chapter 4 in Monoclonal Antibodies: Principles and Applications, Wiley-Liss,

III. Nucleic Acid Sequencing.

In one aspect this invention pertain to the use of redox-active tags in nucleic acid sequencing. Nucleic acid (e.g. DNA) sequencing is generally performed using techniques based on the “chain termination” method described by Sanger et al. (1977) Proc. Natl. Acad. Sci., USA, 74(12): 5463-5467. Basically, in this process, a nucleic acid (e.g. DNA) to be sequenced is isolated, rendered single stranded, and placed into four vessels. In each vessel are the necessary components to replicate the DNA strand, e.g., a template-dependant DNA polymerase, a short primer molecule (e.g. an oligonucleotide) complementary to a known region of the DNA to be sequenced, and individual nucleotide triphosphates in a buffer conducive to hybridization between the primer and the nucleic acid to be sequenced and conducive to chain extension of the hybridized primer (e.g. via a nucleic acid polymerase). In the original Sanger method, each vessel contained a small quantity of one type of dideoxynucleotide triphosphate, e.g. dideoxyandenosine triphosphate(ddA).

In each vessel, each piece of the isolated DNA is hybridized with a primer. The primers are then extended, one base at a time to form a new nucleic acid polymer complementary to the isolated pieces of DNA. When a dideoxynucleotide is incorporated into the extending polymer, this terminates the polymer strand and prevents it from being further extended. Accordingly, in each vessel, a set of extended polymers of specific lengths are formed which are indicative of the positions of the nucleotide corresponding to the dideoxynucleic acid in that vessel. These sets of polymers are then evaluated using gel electrophoresis to determine the template sequence.

The extension fragments are typically evaluated using an electrophoretic procedure (e.g. gel electrophoresis, capillary electrophoresis, chip-based electrophoresis, etc.). The separated nucleic acid fragments are typically identified by detection of a label, in this instantce a redox-active label of this invention. Each species of label corresponds to the incorporation of a particular species of dideoxynucleotide. Thus, label 1 is attached to every extension fragment terminating in a G, label 2 is attached to every extension fragment terminating in a C, label 3 is attached to every extension fragment terminating in an “A”, and label 4 is attached to every extension fragment terminating in a “T” or a “U”.

In a four reaction vessel system, a different species of terminator is placed in each reaction vessel and the fragments in each vessel are labeled with one species of redox-active label. This can be accomplished by the use of a labeled primer, by the use of a labeled terminator, or by the addition of a label and appropriate coupling reagent to the mixture after primer extension.

The sequencing reaction can also be run in a single reaction vessel system. In certain embodiments, all four different elongation terminators (corresponding to A, C, G, and T) are placed in a single reaction vessel. Each terminator is labeled with a different tag, e.g. a redox-active tag according to this invention. When each elongation terminator is incorporated into the growing nucleic acid, elongatioin stops and that nucleic acid fragment is tagged with the label corresponding to the terminator that is incorporated. The fragments can then be separated and detected, e.g. as described above.

Improvements to the original technique described by Sanger et al. have included improvements to the enzyme used to extend the primer chain. For example, Tabor et al. have described enzymes such as T7 DNA polymerase which have increased processivity, and increased levels of incorporation of dideoxynucleotides. (See U.S. Pat. No. 4,795,699 and EP-A1-0 655 506,). More recently, Reeve et al. have described a thermostable enzyme preparation, called THERMO SEQUENASE™, with many of the properties of T7 DNA polymerase. Nature 376: 796-797 (1995). The literature supplied with the THERMO SEQUENASE™ product suggests dividing a DNA sample containing 0.5-2 μg of single stranded DNA (or 0.5 to 5 μg of double stranded DNA) into four aliquots, and combining each aliquot with the THERMO SEQUENASE™ enzyme preparation, one dideoxynucleotide termination mixture containing one ddNTP and all four dNTP's; and a dye-labeled primer which will hybridize to the DNA to be sequenced. The mixture is placed in a thermocycler and run for 20-30 cycles of annealing, extension and denaturation to produce measurable amounts of dye-labeled extension products of varying lengths which are then evaluated by gel electrophoresis.

The processes known for determining the sequence of DNA can be preceded by amplification of a selected portion of the genetic material in a sample to enrich the concentration of a region of interest relative to other DNA. For example, it is possible to amplify a selected portion of a gene using a polymerase chain reaction (PCR), e.g., as described in U.S. Pat. Nos. 4,683,194, 4,683,195 and 4,683,202. In certain embodiments, this process involves the use of pairs of primers, one for each strand of the duplex DNA, that will hybridize at a site located near a region of interest in a gene. Chain extension polymerization (without a chain terminating nucleotide) is then carried out in repetitive cycles to increase the number of copies of the region of interest many times. The amplified polynucleotides are then separated from the reaction mixture and used as the starting sample for the sequencing reaction. In various embodiments, the thermostable enzyme, “Taq polymerase,” derived from the organism Thennus aquaticus is useful in this amplification process (see, e.g., U.S. Pat. Nos. 5,352,600 and 5,079,352).

U.S. Pat. No. 5,427,911 describes a process for coupled amplification and sequencing of DNA. In this process, a sample is combined with two primers and amplified for a number of cycles to achieve 10,000 to 100,000-fold amplification of the initial geneomic DNA. Thereafter, the sample is divided into 8 test and 2 control aliquots. The test aliquots each receive one type of dideoxynucleotide triphosphates and a labeled primer complementary to one of the amplified DNA strands. Thus, the eight test aliquots taken together provide one reaction for each base type in each sequencing direction.

Various nucleic acid sequencing methodlologies are well known to those of skill in the art. Using the teaching and examples provided herein such sequencing methods are easily adapted for use with redox-active labels as described herein.

VIII. Computer-Based Implementation of the Present Invention.

The methods of this invention are readily implemented on various computer systems including, but not limited to stand-alone computers, computers interfaced, e.g. via a data-acquisition interface to a potentiostat, and potentiostats having integrated or associated “dedicated” computer systems.

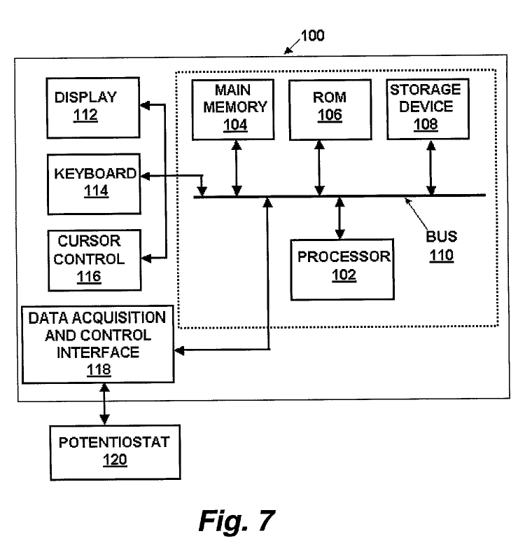

FIG. 7 is a simplified block diagram of a computer system 100 upon which an embodiment of the present invention can be implemented. Computer system 100 includes a bus 110 or other communication medium for communicating information, and a processor 102 coupled to bus 110 for processing information. Computer system 100 further comprises a random access memory (RAM) or other dynamic storage device 104 (referred to as main memory), coupled to bus 110 for storing information and instructions to be executed by processor 102. Main memory 104 can also be used for storing temporary variables or other intermediate information during execution of instructions by processor 102. Computer system 100 also comprises a read only memory (ROM) and/or other static storage device 106 coupled to bus 110 for storing static information and instructions for processor 102. A data storage device 108, such as a magnetic disk or optical disk and its corresponding disk drive, can be coupled to bus 110 for storing information and instructions.

Computer system 100 preferably includes display device 112 coupled to bus 110. Display device can include a cathode ray tube (CRT) or a liquid crystal display (LCD) for displaying information to a computer user. Computer system 100 further includes a keyboard 114 and a cursor control 116, such as a mouse.

Computer system 100 also includes a data acquisition and control interface interface 118 connected to bus 110. Data acquisition and control interface 118 enables computer system 100 to communicate with, record data from and otherwise control potentiostat 120.

The present invention is related to methods of detecting and discriminating particular redox-active labels from a plurality of such labels. As indicated herein, the detection process can involve performing cyclic voltammetry on the label(s) to be detected to produce a cyclic voltammogram for the redox-active labels, and preferably after conversion to the frequency domain, detecting the signal for each redox-active label at a phase angle out of phase with respect to the optimum phase angle for that redox-active label, where a drop-out of signal at said phase angle indicates the presence of the redox-active label. In particularly preferred embodiments, the measurement involves background substraction and/or filtering, transformation of voltammetric data into the time domain (e.g. via Fourier transform), measurement at a particular harmonic and phase angle, and the like.

These various operations, including control of a potentiostat to obtain raw data, can be performed by computer system 100. According to one embodiment, redox-active tag signal detection is performed by computer system 100 in response to processor 102 executing sequences of instructions contained in memory 104. Such instructions can be read into memory 104 from another computer-readable medium, such as data storage device 108, a local network, a wide area network, an internet, and the like. Execution of the sequences of instructions contained in memory 104 causes processor 102 to perform the process steps described herein. In alternative embodiments, hard-wired circuitry may be used in place of or in combination with software instructions to implement the present invention. Thus, the present invention is not limited to any specific combination of hardware circuitry and software. Persons of skill in the art will appreciate that a wide range of hardware and software configurations can support the system and method of the present invention in various specific embodiments.

IV. Kits.

In another aspect, this invention includes kits for conveniently carrying out the methods of the invention. Kits for nucleic acid sequencing typically comprise at least four different and distinguishable redox-active labels as described herein. The kits can additionally include a primer extension reagent, one or more primers (optionally labeled with the redox-active labels), elongation terminators (optionally labeled with the redox-active labels) and the like. In a preferred embodiment, the kits further include a standard template nucleic acid useful for determining the activity of the primer extension reagent.

Other kits include simple labeling kits for attaching or more different redox-active labels to any moiety that it is desired to detect. The redox-active labels can be conveniently derivatized for attachment to a subject moiety.

In still another embodiment, this invention provides a computer readable medium comprising computer readable program code for directing a potentiostat in a cyclic voltammetric measurement to produce a cyclic voltammogram of the redox-active tags; and/or to detecting the signal for each redox-active label it is desired to detect at a phase angle out of phase with the optimum phase angle for said redox-active label, where a drop-out of signal at the phase angle indicates the presence or amount of said redox-active label.

In addition, the kits can, optionally, include instructional materials containing directions (i.e., protocols) for the practice of the methods of this invention. Preferred instructional materials provide protocols utilizing the kit contents for detecting a redox-active label among a plurality of such labels and/or for sequencing nucleic acids. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Capillary Gel Electrophoresis with Sinusoidal Voltammetric Detection: A Strategy to Allow Four-“Color” DNA Sequencing

This example illustrates a novel detection strategy useful in nucleic acid sequencing applications. The approach that utilizes a frequency based electrochemical method is reported. Sinusoidal voltammetry is used to selectively identify four unique redox molecules that are covalently attached to the 5′-end of a 20 base pair sequencing primer. The tags used in this work are ferrocene derivatives with different substituents attached to the ferrocene ring, where the electron donating or withdrawing character of the substituent alters the half-wave potential of the modified ferrocene. Therefore, each tag has a unique SV frequency spectrum that can be easily identified in the frequency domain. In this work, the discrimination of one tag versus all others is accomplished through a “phase-nulling” technique. The signal for each tag is selectively eliminated while the other three responses remain virtually unchanged. This analysis scheme allows for the selective identification of each tagged oligonucleotide eluting in sieving polymer capillary gel electrophoresis with a separation efficiency of 2×10⁶ theoretical plates per meter. This separation efficiency is sufficient to perform nucleic acid sequencing.

Experimental.

Reagents:

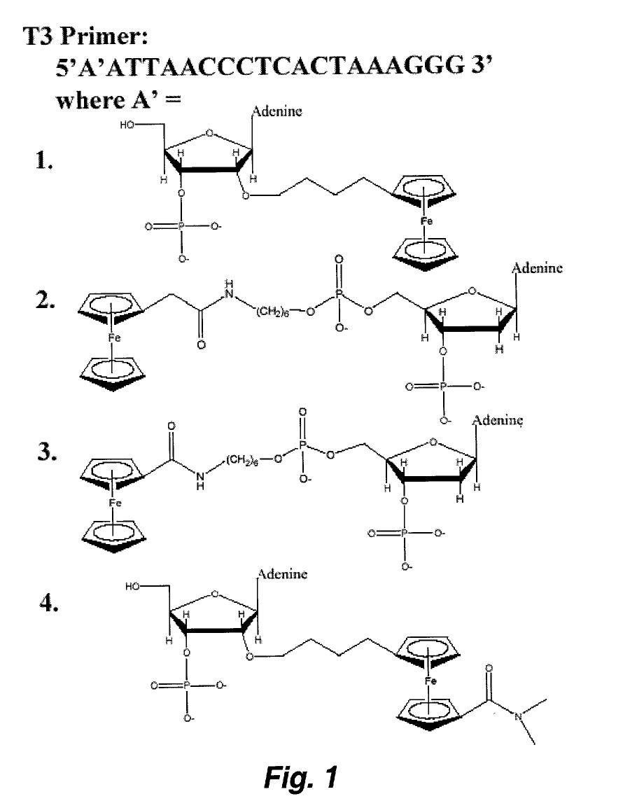

Performance Optimized Polymer-4 (POP-4), Genetic Analyzer Buffer and 5′ amino linked T3 primer (FIG. 1) were all provided by Applied Biosystems (Foster City, Calif.). Two ferrocene phosphoramidites were donated by Clinical Micro Sensors (Pasadena, Calif.). Ferrocene carboxylic acid, ferrocene acetic acid, N-hydroxysuccinimide, and 1,3-dicyclohexylcarbodiimide were used as received (Sigma-Aldrich, Milwaukee, Wis.). Water was deionized and then passed through a Milli-Q water purification system (Millipore Corp., Bedford, Mass.).

Synthesis of 5′ Ferrocene Labeled Primers:

The synthetic procedure of Ihara et al. (Ihara et al. (1996) Nucleic Acids Research, 24: 4273-4280; Ihara et al. (1997) Chemical Communications, 1609-1610) was followed for the synthesis of both the ferrocene carboxylic acid (FC) and ferrocene acetic acid (FA) labeled oligonucleotides. Briefly, an activated N-hydroxysuccinimide (NHS) ester of ferrocene carboxylic acid and ferrocene acetic acid was synthesized using NHS and dicyclohexylcarbodiimide. The product was purified on a column of silica gel (Merck 60, methylene chloride eluent). The FC synthesis produced a yellowish-orange solid with a yield of 65% and the FA synthesis produced a reddish-brown solid with a yield of 45%. The activated ester product was confirmed by ¹H-NMR (DMSO).

The activated ester (dissolved in dimethyl sulfoxide) was then linked to the 5′ amine terminated T3 primer in a 0.5 M NaHCO₃/Na₂CO₃ (pH=9) buffer solution overnight. The reaction mixture was diluted to 1 ml with water and chromatographed on a NAP-10 column (Pharmacia Sephadex G-25). The absorbance was measured at 260 nm and used to determine which fractions contained DNA. Those fractions were combined, lyophilized, and purified by RP-HPLC.

HPLC Purification of Oligonucleotides:

The HPLC system consisted of a L-6200A Intelligent pump (Hitachi), a 100 μL sample loop, and an SSI 500 variable UV/Vis detector (Spectra-Physics, San Jose, Calif.). The HPLC conditions were as follows: 25° C., 0.5 ml/min flow rate, buffer A was 0.1 M TEAA (pH=7), and buffer B was acetonitrile. A linear gradient was applied from 10% to 40% B in 30 minutes. The column used was a Thompson Liquid Chromatograph 100, C-18 with 5 um particles, 4.6 mm i.d. and 15 cm in length. The retention times for the two modified oligonucleotides were 13 min. and 15 min. for the FC and FA labeled primers respectively. The identity of these compounds was confirmed by MALDI-TOF MS, FA m/z found was 6496±6 (calculated m/z=6500) and FC m/z found was 6482±6 (calculated m/z =6486) (Voyager DE-STR, PerSeptive Biosystems, Foster City, Calif.; see supporting information).

Preparation of FC-Labeled PCR Products:

Longer DNA fragments were generated by PCR using the FC-tagged T3 forward primer. Fragments of 159 or 268 base pairs were generated by using appropriate unlabeled reverse primers (see Supplemental Information) on the pBluscript SK+plasmid. For PCR, 660 ng of each primer, 100 ng of pBSK+, 2 μL each of 10 mM solutions of A, T, G, and C, 1 μL of 100 mM MgCl₂, 10 μL of PCR buffer, and 0.5 μL of Taq polymerase (5 units/μL) were used in a 100 μL reaction volume. Thermocycling was performed in an ABI GeneAmp PCR System 2400 as follows: denaturation at 940 C for 5 min, followed by 40 cycles of 94° C. for 30s, 41° C. for 30s, and 72° C. for 60 s, then held at 72° C. for 4 minutes at the end of cycling. The individual PCR products could be separated and identified with gel electrophoresis (data not shown), demonstrating that the ferrocene tag does not interfere with the PCR reaction.

Carbon Cylinder Microelectrodes:

The fabrication of carbon cylinder microelectrodes has been described previously (Pantano, P.; Kuhr, W. G. Anal. Chem. 1993, 65, 623-630). Briefly, 32 μm carbon fibers were aspirated into glass capillaries and pulled with a microelectrode puller (model PE-2, Narishige, Tokyo Japan). The pulled end of the glass capillary was then cut under a microscope with a scalpel. The end was sealed with epoxy in one of two ways: either the glass capillary was backfilled with epoxy (EPO-TEK 314, Epoxy technology, Billerica, Mass.) followed by insertion of a copper wire or the pulled end was exposed to a small drop of epoxy which wicked up creating the seal. In the latter case, electrical contact with the carbon fiber was made by backfilling the glass capillary with gallium tin eutectic (Sigma Chemical Corp.). The carbon cylinder was clipped to 100 μm or less with a scalpel and sonicated in water prior to use.

Capillary Gel Electrophoresis:

Capillary gel electrophoresis was performed with a battery powered high voltage supply. The power supply was built with a 12 V rechargeable battery and a G50 HV module (EMCO High Voltage Corporation, Sutter Creek, Calif.). The power supply is capable of supplying either +/−5000 Volts. The capillary used was 360 μm o.d.×20 μm i.d. bare fused silica capillary (Polymicro Technologies, Phoenix, Ariz.) with a length of 19 cm between the injection and detection end. The detection end of the capillary was etched with 40% hydrofluoric acid to produce a slightly larger inner diameter, thus allowing the carbon fiber cylinder electrode to be placed just inside the end of the capillary. The capillary was dynamically coated with POP-4 sieving matrix by pumping it through the capillary (100 psi) for 10-15 minutes and subsequently conditioned with the electrophoresis buffer, Genetic Analyzer Buffer (1×), under an electric field for 15 minutes.

Flow Injection Analysis:

The FIA system consisted of a 434 μm o.d.×324 μm i.d fused silica capillary (Polymicro Technologies) 23.5 cm in length and mounted onto a three dimensional stereotaxic manipulator (Kopf Instruments, Tujunga, Calif.). The flow rate and sample injection were controlled by gravity; the buffer reservoir was positioned 1 cm above the detection cell resulting in a flow rate of 19 μl/min. The micropositioner was used to position the carbon cylinder electrode into the end of the FIA capillary. The detection reservoir was constructed by suspending a drop of buffer on top of a vial, which was placed directly under the outlet of the capillary/electrode assembly. The Ag/AgCl (saturated KCl) reference electrode was placed in the detection vial. Electrochemical detection in the CE experiments used the same detection vial and micropositioner to manipulate the carbon cylinder electrode just inside (5-10 μm) the etched capillary end. The four electrochenically-tagged primers were injected sequentially into the FIA capillary; the data for each of the four sample plugs was collected individually and then all data sets were combined into one large data set prior to data analysis.

Cyclic Voltammetry:

The cyclic voltammetry (CV) experiments were run with a program written in house utilizing Labview software (National Instruments, Austin, Tex.). The excitation and acquisition was performed with a 16-bit data acquisition card (PCI-445 1, National Instruments). Cyclic voltammetry was performed using a scan rate of 1 V/s scanning from −200 to 650 mV. The triangle waveform was applied to and collected from the electrochemical cell with a 2-electrode potentiostat (Geneclamp 500, Axon Instruments, Foster City, Calif.). After the signal from the working electrode was collected by the potentiostat it was passed through a 4-pole low pass Bessel filter (Cyberamp 380, Axon Instruments) set at 40 Hz. The same potentiostat and filter system was used to run sinusoidal experiments as well (Hayes et al. (1998) Biosensors & Bioelectronics, 13: 1297-1305).

Sinusoidal Voltammetry:

The experimental approach for data acquisition in the SV experiment has been described previously (Brazill et al. (2000) Anal Chem. 72: 5542-5548). Briefly, a computer-generated sine wave (PCI-2251, National Instruments, Austin, Tex.) scans through the potential window of interest. In this work, the excitation waveform used an 11 Hz sine wave scanning between −200 to +650 mV or −200 to +800 mV vs. Ag/AgCl (saturated KCl), allowing for the detection of all four ferrocene labeled primers. The 16-bit data was acquired (PCI-4451, National Instruments) at 128 times the excitation frequency (1408 Hz) and each scan consisted of 4 cycles of 512 points. Frequency domain information was obtained by continuous conversion of each scan of 512 points via the application of a Fast Fourier Transform (FFT) in a program written in Labview (National Instruments); only the magnitude and phase information corresponding to the first ten harmonics of the excitation were saved to disk.

This frequency domain data provides voltammetric information characteristic of each redox active species (the SV frequency spectrum) and time course information (current versus time), as described previously (Brazill et al. (2000) Anal. Chem. 72: 5542-5548). The frequency spectrum for a specific analyte consists of a series of vectors (represented as magnitude and phase) at each of the ten harmonics. To isolate the information particular to a given analyte, a background frequency spectrum is subtracted from the entire data set. The background spectrum is defined typically as the average of the first ten scans, which represent all the background processes (capacitive and faradaic) at the electrode surface (in the absence of analyte). The frequency spectrum for the analyte of interest is simply defined as the background-subtracted current vector (magnitude and phase) at the highest part of the signal.

In addition to the SV frequency spectrum, time course data can be obtained at each harmonic frequency element by performing the “digital equivalent of a lock-in amplifier” (Brazill et al. (2000) Anal. Chem. 72: 5542-5548). Analogous to an analog lock-in amplifier, the instantaneous current is monitored at the optimum phase angle for the signal of interest, thus increasing the sensitivity and selectivity over traditional voltammetric techniques. Maximum sensitivity is achieved if the background phase angle is 90 degrees out of phase with the optimum phase angle for the signal. Therefore, the analyte is typically monitored at the phase angle that gives the maximum current for the signal and minimum background, which increases the S/N for the measurement. Finally, the phase-optimized time course data was digitally filtered with a low-pass filter using a boxcar averaging routine.

The digital lock-in approach can also be used to distinguish between different molecules in the time domain (3razill et al. (2000) Anal. Chem. 72: 5542-5548). Selective discrimination between analytes with different electrochemical characteristics (formal potential, kinetics, etc.) is accomplished by identifying the frequency component where signal due to the analyte is closest to 90° out of phase with all other components. Careful selection of the frequency and phase angle allows one to isolate the signal of the component of interest in almost any complex matrix.

Results and Discussion:

The development of four unique redox active molecules that can be used in cycle sequencing reactions is analogous to current sequencing schemes that use four different fluorophores as the tags. The tags used in this work are based on ferrocene derivatives, which are attached to either the 5′ end of a 20-mer primer or incorporated into the deoxynucleotide triphosphate used to synthesize the primer, FIG. 1. In order to validate the potential use of a ferrocene-tagged primer in DNA sequencing reactions, the tagged oligomer should preferably be based on a commonly used primer. In this case, the sequence is the “T3 primer” which can be used to sequence many Bluescript-based vectors and other plasmids. The pBluescript SK+ plasmid was chosen to test the compatibility of the ferrocene-tagged T3 primer, since this is a high copy number plasmid commonly used for DNA sequencing that generally provides large yields. A ferrocene-tagged T3 forward primer can be used to generate a number of PCR fragments by using appropriate unlabeled reverse primers on the pBluescript SK+plasmid (Mayer (1995) Gene, 163: 41-46).

Voltammetry of Redox-Tagged Oligonucleotides.

Each electrochemical tag, which codes for a specific base, should have a unique voltammetric response. Ferrocene derivatives are ideal redox tags because of their chemical stability as well as their fast, reversible electrochemical kinetics. Moreover, it has been shown that the half-wave-potential of the molecule can be adjusted to various potentials by placing electron withdrawing or donating groups on the ferrocene ring (Emilia et al. (1994) J. Organometallic Chemistry, 480: 81-90). In this manner, we found four ferrocene tags that have significantly different E_{1/2}'s by virtue of the difference in their substituent groups attached to the cyclopentadienyl ring (FIG. 1: AF: alkyl ferrocene, FA: ferrocene acetate, FC: ferrocene carboxylate, and AFD: alkyl ferrocene dimethylcarboxamide).

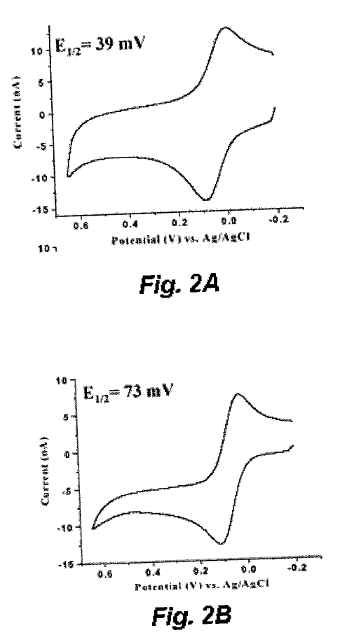

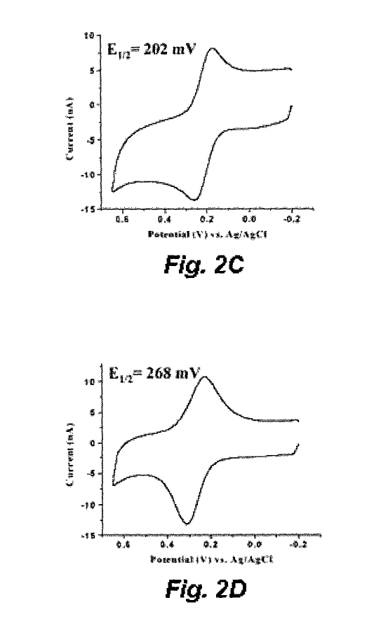

Cyclic voltammetry of all four ferrocene-tagged T3 primers was performed in a solution of GA buffer (1×) at a concentration of 100 μM, as shown in FIG. 2. The electrochemical cell consisted of a 32 μm-diameter carbon fiber microelectrode and an Ag/AgCl wire reference electrode inserted into a minimum volume of each solution (2-25 μL). The CV's were obtained at 1 V/s from −200 to 650 mV for all four ferrocene-tagged primers. Each of the ferrocene-tagged T3 primers has a different formal potential verse the Ag/AgCl reference electrode; CV's of the AF T3 primer (E_{1/2}=39 mV vs. Ag/AgCl), the FA T3 primer (E_{1/2}=73 mV vs. Ag/AgCl), the FC T3 primer (E_{1/2}=202 mV vs. Ag/AgCl), and the AFD T3 primer (E_{1/2}=268 mV vs. Ag/AgCl) are shown in FIGS. 2A-2D, respectively. The ΔEₚₑₐₖ for each tagged primer is close to 60 mV, which indicates reversible Nernstian behavior. The lack of significant peak splitting indicates that the incorporation of the ferrocene derivative into a large molecule, a 20-mer oligonucleotide, does not significantly alter the observed kinetics of ferrocene oxidation. As shown in FIG. 2, the voltammetry of the four oligonucleotides tagged with unique ferrocene derivatives are different, but significantly overlap one another. The difference in redox properties between the tags in FIG. 2 is clearly evident, but it would be difficult to independently identify each individual species with traditional voltammetric methods, especially in a CGE separation, due to limitations in the ability of these methods to discriminate molecules with higher oxidation potentials from those that are easier to oxidize.

Sinusoidal Voltammetry of Redox-Tagged Oligonucleotides.

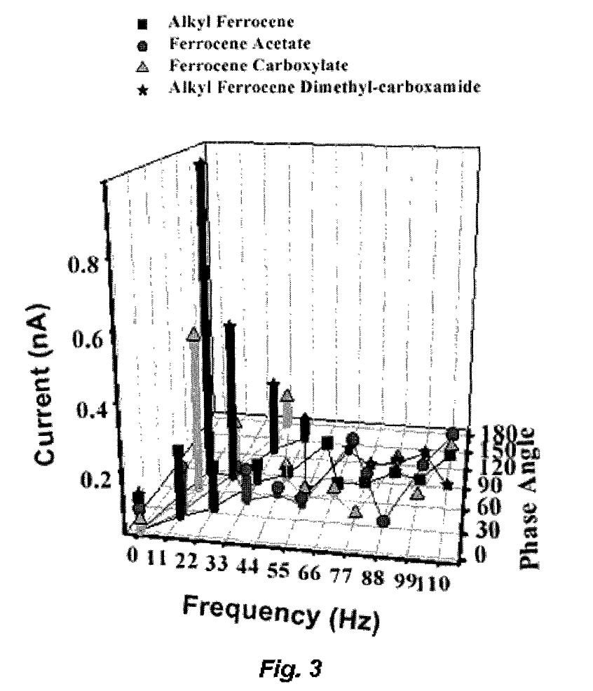

Sinusoidal voltammetry is capable of providing sufficient selectivity to identify each individually tagged oligonucleotide in the frequency domain by producing an electrochemical response specific to each electrochemical tag, which in turn, codes for each base. FIG. 3 shows the SV frequency domain spectra for each of the four unique electrochemically-tagged oligonucleotides. The tagged oligonucleotides were sequentially injected into the FLA system at a concentration of 10 μM in GA buffer (lx) and the SV response was collected for each injection. The data in FIG. 3 represents the background-subtracted current response, both magnitude and phase, obtained at the signal maximum for each ferrocene-tagged T3 primer (see FIG. 4). As shown, each ferrocene-tagged oligonucleotide is easily identified in the frequency domain using this approach. Examination of the phase response for each tag allows the identification of the frequency component that is most unique. Clearly, the third harmonic (33 Hz) shows the biggest difference in phase angle between all ferrocene-tagged oligonucleotides under these conditions.

Phase-Common and Phase-Nulled Time-Course Information.

By using frequency domain information, it is quite simple to find a specific frequency and phase angle that is selective for an individual tag (see below). However, it would be fairly difficult to find a set of frequencies and phase angles that can selectively identify one signal while eliminating the response from three other tags. This problem, i.e., the identification of individual components in the separation and elution of a complex mixture, requires a more subtle solution. One such solution, which we will call “phase-nulling”, involves using the digital lock-in algorithm to specifically eliminate the response of the component of interest. When the time course of the phase-nulled signal is compared to the response at a phase angle where all components are present (the “phase-common” signal), the component of interest is easily identified by its absence (FIG. 4). We have demonstrated that complex background signals can be effectively “nulled” by finding the phase angle of the background and monitoring at a phase angle 90° out of phase with this signal. One real advantage of this approach is that it utilizes exactly the same data set for each analysis, such that each response will be exactly aligned in time with all others.

The first step in the generation of selective time-course information is to find a frequency and phase angle where all components can be monitored with similar sensitivity (i.e. monitor at a single frequency and phase angle where each tag is equally represented). This “phase-common” signal can be obtained at any given frequency, simply by examining the difference between signals as a function of phase angle. The variance between each pair of components is calculated at each phase angle (e.g., i_{ab}=(iₐ−i_{b})²), and the overall variance (i_{TOTAL}=Σiᵢ/n, where iᵢ are the individual variances and n is the total number of combinations), representing the sum of all the individual variances, is calculated and minimized to find the optimum phase angle for all components. This allows rapid identification of a single phase angle that can be used to monitor all four tags with only slight variation from their maximum current response.

Application of this procedure to the FIA SV frequency spectra shown in FIG. 3 produces a phase-common signal with a phase angle of 160 degrees at the third hannonic. Under these conditions, all four tags are monitored with similar amplitudes (FIG. 4A). A large signal is observed for all four tags, with minimal loss in signal compared to that observed at each of the optimum phase angles (data not shown). The phase-nulled signals for each redox-tagged oligonucleotide are shown in FIGS. 4B-4E. This data was obtained at the same frequency (third harmonic) by locking-in at a phase angle that is exactly 90 degrees out of phase from the optimum for each tag. The phase angle can be either +or −90 degrees out of phase, it does not make a difference. As shown in each trace, the signal corresponding to the phase-nulled component is effectively diminished while the other three tags are relatively unaffected. When compared to the phase-common signal (FIG. 4A), it is easy to see which peak has been removed. Therefore, this method should allow rapid base calling on a single set of data simply by identifying the peaks that disappear.

Independence of Phase Angle on Concentration and Length of Oligonucleotide.

In order for this methodology to be applicable to sequencing and fingerprint analysis, the phase angles of the individual tags are preferably independent of analyte concentration as well as length of the attached oligonucleotide. This facilitates the use of the “phase-nulling” scheme to identify the tags as they elute out of a separation capillary. The precision in phase angle of different analyte concentration is demonstrated in Table 1 (column 2).

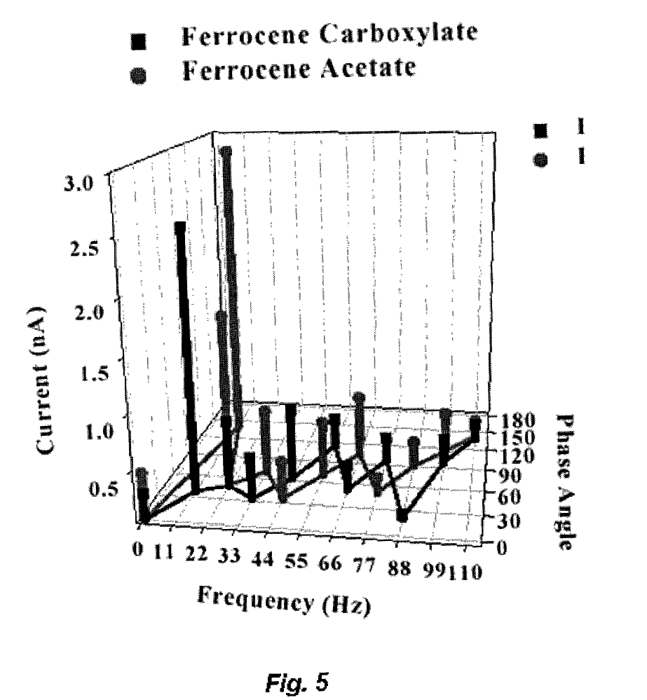

TABLE 1

| | | | FC-tagged 268- |
|---|---|---|---|
| | FC-tagged 20-mer | | mer |
| Frequency | Phase angle (degree) ± | FC-tagged 159-mer | Phase angle |
| (Hz) | standard error (n = 9) | Phase angle (degree) | (degree) |
| 11 | 42 ± 1 | 44 | 46 |
| 22 | 49 ± 3 | 47 | 53 |
| 33 | 30 ± 4 | 37 | 36 |
| 44 | 59 ± 2 | 57 | 58 |
| 55 | 117 ± 5 | 92 | 104 |
| The data in this table represent the optimum phase angles measured for FC-tagged 20-mer (T3 primer), FC-tagged 159 base pair PCR fragment, and FC-tagged 268 base pair PCR fragment. Column 2 represents the aggregate average of the phase angles measured for six different concentrations of FC-tagged T3 primer ranging in concentration between 100 nM to 10 μM. | | | |
| # Column 3 and 4 were obtained with a concentration of approximately 1 μM DNA in each case. The data in the table were obtained under the same electrochemical conditions as in FIG. 5. | | | |

The data in this table represent the optimum phase angles measured for FC-tagged 20-mer (T3 primer), FC-tagged 159 base pair PCR fragment, and FC-tagged 268 base pair PCR fragment. Column 2 represents the aggregate average of the phase angles measured for six different concentrations of FC-tagged T3 primer ranging in concentration between 100 nM to 10 μM. Column 3 and 4 were obtained with a concentration of approximately 1 μM DNA in each case. The data in the table were obtained under the same electrochemical conditions as in FIG. 5.

The data represents the average phase angle±the standard error obtained in 9 replicate injections of the FC T3 primer at different concentrations. The concentration ranges over three orders of magnitude from 100 nM to 10 μM. The average phase angle±standard error for the first 5 harmonics are shown (since they are the ones most commonly employed in this work), and the standard error reported is essentially the same magnitude as the experimental error for the measurement of replicate injections of the same sample. This demonstrates that the phase angle is independent of analyte concentration and is an intrinsic property of the electrochemical characteristics of the analyte signal.

Likewise, for this to work in a sequencing application, it is preferably that the phase angle of a tag remain consistent and independent of the length of DNA that it is attached to. We examined the effect of the length of the oligonucleotides by generating PCR fragments of 159 bases and 268 bases using FC-tagged T3 as the forward primer. The data in Table 1 (column 3 and 4) gives the phase angles for the FC tag attached to a 159 mer and a 268 mer. The consistency in phase angle with the FC T3 primer for the first five harmonics indicates that the length of the DNA that the tag is attached to does not seem to alter the electrochemical properties of the FC tag significantly. Therefore, the “phase-nulling” approach should work effectively regardless of the length of fragment or concentration of the electrochemical tag.

Sinusoidal Voltammetry of Ferrocene-Tagged Oligonucleotides in Capillary Gel Electrophoresis.

In order to demonstrate that this technique can be used in DNA analysis it was necessary to couple the SV detection to a CGE system. The first experiment we performed was to ensure that the electrochemical properties of the FA and FC-tagged T3 primers did not change significantly under electrophoresis conditions. The CV of these two tags under an electric field and with the carbon cylinder exposed to the sieving matrix (POP-4) did not produce significant peak splitting. The E_{1/2} for each molecule was shifted slightly by approximately ±15 mV (data not shown). The role of the CE current on the shift in half-wave potentials was investigated by Wallenborg et al. (1999) Anal. Chem., 71: 544-549, but was not investigated thoroughly herein. We note the CV's under CGE conditions were very consistent with the static voltammograms shown in FIG. 2.

The FA and FC tagged T3 primers were then injected into the CGE capillary and their respective SV frequency spectra were collected (FIG. 5). A 100 nM FC T3 primer solution was injected first for 12 seconds at −174 V/cm, and then a 100 nM solution of FA T3 primer was injected for 12 seconds at −174 V/cm. At this point, the capillary was placed in the separation buffer and the electrophoretic separation was continued at −305 V/cm. This allowed for temporal separation of the two identical 20-mer primers labeled with different tags. We utilized a commercially available sieving matrix polymer, POP-4, because it is specifically designed for high-resolution fragment analysis. POP-4 is capable of separating fragments that differ in size by 1 base up to 250 nucleotides in about 30 minutes (Wenz et al. (1998) Genome Research 8: 69-80). An 11 Hz sine wave with a potential window of −200 to 800 mV vs. Ag/AgCl (sat'd KCl) was applied to the electrochemical cell. By using this frequency domain information, it is quite simple to find a frequency and phase angle that is selective for each tag, such that the response for the tag of interest is easily eliminated.

Phase-Selective SV Time-Course Data in Capillary Gel Electrophoresis.

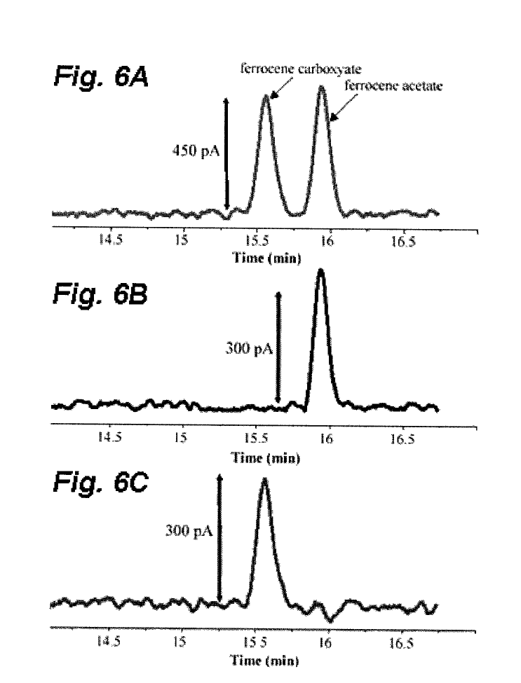

Application of the phase-common and phase-nulling procedure to the CGE SV frequency spectra shown in FIG. 5 produces a phase-common signal with outstanding signal quality at the third harmonic (33 Hz at 36 degrees). Under these conditions, both tags are monitored with similar amplitudes relative to their optimum phase angle responses (FIG. 6A). In this case, the two tags have such different electrochemical properties (e.g., FIG. 5) that it is easy to find a frequency and phase angle to produce phase-nulled signals for each redox-tagged oligonucleotide, as shown in FIGS. 6B and 6C. This data was obtained at the third harmonic by locking-in at a phase angle that is exactly +90 degrees out of phase for the FC and FA tagged T3 primers (123 and 161 degrees, respectively). As shown in each trace, the signal corresponding to the phase-nulled component is effectively diminished while the other tag is still present. When compared to the phase-common signal (FIG. 6A), it is easy to see which peak has been removed. The signal/noise of these measurements indicate that a detection limit of 900 pM can be extrapolated (S/N=3). It is important to note that the half-width of each peak in this separation is less than 6 seconds, corresponding to a separation efficiency of 2×10⁶ theoretical plates per meter. This separation efficiency is sufficient to perform DNA sequencing; the conditions used in this work have can be further optimized for high-resolution sequencing applications.

Conclusions

Electrochemical detection coupled with capillary separations offers many advantages including simplicity, inexpensive instrumentation, and compatibility with miniaturization. In this work, sinusoidal voltammetry was used to selectively monitor four unique ferrocene-labeled primers and a novel sequencing detection system was demonstrated. We have shown that each of the four tags can be selectively nulled, leading to the identification of each fragment for base calling simply by noting those peaks that disappear from the original data set. This approach utilizes a single data set as well as a single harmonic to identify each of the four different tags. It was also demonstrated that this approach is transferable to the CGE format. The discrimination of two of the tagged primers is demonstrated while under an electric field and exposed to a sieving matrix with a separation efficiency sufficient to perform DNA sequencing.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of determining the sequence of a nucleic acid template, said method comprising:
i) generating and redox labeling sets of complementary sequencing fragments of said template where the sets of fragments terminating with the four different bases A, C, G, or T are each label labeled with a redox-active label that has an oxidation state distinct and distinguishable from the redox states of the labels labeling the other sets of fragments;
ii) separating said sequencing fragments;
iii) performing cyclic voltammetry on said sequencing fragments to produce a cyclic voltammogram for the redox-labeled sequencing fragments;
iv) detecting the signal for each redox-active label at a phase angle out of phase with respect to the optimum phase angle for said redox-active label, where a drop-out of signal at said phase angle indicates the presence of said redox-active label.

2. The method of claim 1, wherein said dropout is as compared to the signal present at the phase common signal.

3. The method of claim 1, wherein said fragments are generated with a termination method employing primers, and terminators, and the primers or the terminators are labeled with said redox-active labels.

4. The method of claim 3, wherein said fragments are generated with dideoxy terminators.

5. The method of claim 4, wherein said fragments are generated with dideoxy terminators selected from the group consisting of 2′,3′-dideoxyguanosine-5′-triphosphate, 7-deaza-2′,3′-dideoxyguanosine-5′-triphosphate, 2′,3′-dideoxyadenosine-5′-triphosphate, 2′,3′-dideoxythymidine-5′-triphosphate, and 2′,3′-dideoxycytidine-5′-triphosphate.

6. The method of claim 1, wherein nucleoside triphosphates used for chain elongation are labeled with said redox-active labels.

7. The method of claim 1, wherein said redox-active labels are independently selected from the group consisting of a porphyrin, an expanded porphyrin, a contracted porphyrin, a metallocene, a linear porphyrin polymer, and a porphyrin array.

8. The method of claim 7, wherein said redox-active labels comprise a ferrocene.

9. The method of claim 8, wherein said ferrocene is selected from the group consisting of an alkyl ferrocene, a ferrocene acetate, a ferrocene carboxylate, and an alkyl ferrocene dimethylcarboxamide.

10. The method of claim 1, wherein said redox-active labels comprise a porphyrinic macrocycle substituted at β-position or at a meso-position.

11. The method of claim 1, wherein said voltammetry is performed at a single electrode.

12. The method of claim 1, wherein said voltammetry utilizes a sinusoidal waveform.

13. The method of claim 1, wherein said cyclic voltammetry comprises converting voltammetric data into a time or frequency domain to provide a frequency spectrum for a redox-active label.

14. The method of claim 12, wherein said cyclic voltammetry comprises converting voltammetric data into a time or frequency domain to provide a frequency spectrum for a redox-active label.

15. The method of claim 13, wherein said converting comprises performing a Fourier transform.

16. The method of claim 13, wherein said cyclic voltammetry comprises selecting voltammetric data at a second or higher harmonic frequency.

17. The method of claim 16, wherein said cyclic voltammetry comprises selecting voltammetric data at a third or higher harmonic frequency.

18. The method of any one of claims 1, 13, or 16, wherein said cyclic voltammetry comprises selecting voltammetric data at a phase angle about 45 degrees to about 90 degrees out of phase with the optimum phase angle for the redox-active label whose presence is to be detected.

19. The method of claim 18 wherein said cyclic voltammetry comprises selecting voltammetric data detecting at a phase angle closest to 90 degrees out of phase with the optimum phase angle for the redox-active label whose presence is to be detected.

20. The method of claim 1, wherein separating said sequencing fragments comprises electrophoretically separating said sequencing fragments.

21. The method of claim 1, wherein separating said sequencing fragments comprises chromatographically separating said sequencing fragments.

22. A chain-termination type nucleic acid sequencing method, said method comprising:
i) providing a template nucleic acid;
ii) annealing an oligonucleotide primer to a portion of said template nucleic acid thereby forming a primer-template hybrid;
iii) adding a primer-extension reagent to the primer-template hybrid for extending the primer and forming a primer extension product, the primer extension reagent comprising nucleoside triphosphates; and
iv) adding a terminator to the primer-template hybrid for causing specific termination of the primer extension and formation of a plurality of primer extension products where said terminator or said oligonucleotide primer is labeled with one of four redox-active tags where said redox-active tags have different and distinguishable oxidation states;
v) separating said primer extension products; and
vi) detecting the signal for each redox-active label at a phase angle out of phase with the optimum phase angle for said redox-active label, where a drop-out of signal at said phase angle indicates the presence of said redox-active label.

23. A method of detecting a tagged analyte, said method comprising:
i) providing at least two species of tagged analyte
ii) performing cyclic voltammetry on said tagged analytes to produce a cyclic voltammogram for said tagged analytes;
iii) detecting the signal for a redox-active label at a phase angle out of phase with the optimum phase angle for said redox-active label, where a drop-out of signal at said phase angle indicates the presence of said redox-active label.

24. The method of claim 23, wherein said providing comprises providing at least four species of tagged analyte where each species of tagged analyte is tagged with a redox-active label where the redox-active label attached to each species has an oxidation state different and distinguishable from the oxidation states of the redox-active labels attached to the other species of tagged analyte.

25. The method of claim 23, wherein said redox-active label is selected from the group consisting of a porphyrinic macrocycle, a metallocene, a linear polyene, a cyclic polyene, a heteroatom-substituted linear polyene, a heteroatom-substituted cyclic polyene, a tetrathiafulvalene, a tetraselenafulvalene, a metal coordination complex, a buckyball, a triarylamine, a 1,4-phenylenediamine, a xanthene, a flavin, a phenazine, a phenothiazine, an acridine, a quinoline, a 2,2′-bipyridyl, a 4,4′-bipyridyl, a tetrathiotetracene, and a peri-bridged naphthalene dichalcogenide.

26. The method of claim 23, wherein said redox-active label is selected from the group consisting of a porphyrin, an expanded porphyrin, a contracted porphyrin, a metallocene, a linear porphyrin polymer, and a porphyrin array.

27. The method of claim 26, wherein said redox-active labels comprise a ferrocene.

28. The method of claim 27, wherein said ferrocene is selected from the group consisting of an alkyl ferrocene, a ferrocene acetate, a ferrocene carboxylate, and an alkyl ferrocene dimethylcarboxamide.

29. The method of claim 23, wherein said redox-active label comprises a porphyrinic macrocycle substituted at a β-position or at a meso-position.

30. The method of claim 23, wherein said voltammetry is performed at a single electrode.

31. The method of claim 23, wherein said cyclic voltammetry utilizes a sinusoidal excitation waveform.

32. The method of claim 23, wherein said cyclic voltammetry comprises converting voltammetric data into a time or frequency domain to provide a frequency spectrum for a redox-active label.

33. The method of claim 32, wherein said converting comprises performing a Fourier transform.

34. The method of claim 32, wherein said cyclic voltammetry comprises selecting voltammetric data at a second or higher harmonic frequency.

35. The method of claim 44, wherein said cyclic voltammetry comprises selecting voltammetric data at a third or higher harmonic frequency.

36. The method of any one of claims 23, 32, 34, or 35 wherein said cyclic voltammetry comprises selecting voltammetric data at a phase angle about 45 degrees to about 90 degrees out of phase with the optimum phase angle for the redox-active label that is to be detected.

37. The method of claim 36 wherein said cyclic voltammetry comprises selecting voltammetric data detecting at a phase angle about 90 degrees out of phase with the optimum phase angle of the redox-active label that is to be detected.

38. The method of claim 23, wherein said analytes are selected from the group consisting of nucleic acids, proteins, and antibodies.

39. The method of claim 23, wherein said redox-active label is attached to a chain terminator.

40. The method of claim 23, wherein said redox-active label is attached to a nucleic acid.

41. The method of claim 23, wherein said analytes are electrophoretically separated nucleic acids.

42. The method of claim 23, wherein said analytes are chromatographically separated nucleic acids.

43. The method of claim 23, wherein said providing comprises providing four species of tagged analyte where each species of tagged analyte is tagged with one of four different and distinguishable redox-active label.

44. A method of selective electrochemical detection of analytes in a complex mixture of analytes, said method comprising:
i) labeling each analyte in the mixture with a redox label that generates an electrochemical signal that is different from the labels attached to other analytes in said mixture where said labeling provides labeled analytes;
ii) performing cyclic voltammetry on said labeled analytes to produce a cyclic voltammogram for said labeled analytes;
iii) detecting the signal for a redox-active label at a phase angle out of phase with the optimum phase angle for said redox-active label, where a drop-out of signal at said phase angle indicates the presence of said redox-active label.

45. A computer-readable medium that can be used for directing an apparatus to detect and distinguish a plurality of redox-active tags where said redox-active tags have different and distinguishable oxidation states, said computer readable medium comprising;
computer readable program code for directing a potentiostat in a cyclic voltammetric measurement to produce a cyclic voltammogram of said redox-active tags;
computer readable program code for detecting the signal for each redox-active label at a phase angle out of phase with the optimum phase angle for said redox-active label, where a drop-out of signal at said phase angle indicates the presence or amount of said redox-active label.

46. The computer readable medium of claim 45, wherein said plurality of redox-active tags comprises four redox-active tags.

47. The computer readable medium of claim 45, wherein said cyclic voltammetric measurement is performed at a single electrode.

48. The computer readable medium of claim 45, wherein said cyclic voltammetric measurement is sinusoidal voltammetry.

49. The computer readable medium of claim 45, wherein said code for detecting the signal comprises code for converting voltammetric data into a time or frequency domain to provide a frequency spectrum for a redox-active label.

50. The computer readable medium of claim 49, wherein said converting comprises performing a Fourier transform.

51. The computer readable medium of claim 45, wherein said code for detecting the signal comprises code for selecting voltammetric data at a second or higher harmonic frequency.

52. The computer readable medium of claim 51, wherein said code for detecting the signal comprises code for selecting voltammetric data at a third or higher harmonic frequency.

53. The computer readable medium of any one of claims 46, 51, or 52, wherein said code for detecting the signal comprises code for selecting voltammetric data at a phase angle about 45 degrees to about 90 degrees out of phase with the optimum phase angle for said redox-active label.

54. The computer readable medium of claim 53, wherein said code for detecting the signal comprises code for selecting voltammetric data detecting at a phase angle about 90 degrees out of phase with the optimum phase angle for said redox-active label.

55. The computer readable medium of claim 45, wherein said computer readable medium is selected from the group consisting of a magnetic disk, an optical disk, and a chip.

56. The computer readable medium of claim 45, wherein said computer readable medium is a component of a nucleic acid sequencer.

57. A computer-readable storage medium storing program code for causing a computer to detect and distinguish a plurality of redox-active tags where said redox-active tags have different and distinguishable oxidation states, said computer readable medium comprising program code directing a computer to:
detect the signal for each redox-active label at a phase angle out of phase with the optimum phase angle for said redox-active label, where a drop-out of signal at said phase angle indicates the presence or amount of said redox-active label.

58. The computer readable storage medium of claim 57, wherein said computer readable medium further comprises program code for directing a potentiostat in a cyclic voltammetric measurement to produce a cyclic voltogram of said redox-active tags.

59. A kit for sequencing a nucleic acid, said kit comprising:
four redox-active tags wherein said redox active tags have different and distinguishable oxidation states; and
instructional materials teaching the detection of the signal for each redox-active label at a phase angle out of phase with the optimum phase angle for said redox-active label, where a drop-out of signal at said phase angle indicates the presence or amount of said redox-active label.

60. The kit of claim 59, wherein said redox-active labels are attached to elongation terminators.

61. The kit of claim 60, wherein said elongation terminators are dideoxy elongation terminators.

62. A kit for sequencing a nucleic acid, said kit comprising:
a plurality of redox-active tags where said redox active tags have different and distinguishable oxidation states; and
a computer readable medium of claim 45.

63. The kit of claim 62, wherein said kit comprises four or more redox-active labels.

64. A kit for sequencing a nucleic acid, said kit comprising:
a plurality of redox-active tags where said redox active tags have different and distinguishable oxidation states; and
a computer readable medium of claim 57.

65. The kit of claim 64, wherein said kit comprises four or more redox-active labels.

66. In a computer system containing stored software programs, a method of detecting a tagged analyte from a plurality of tagged analytes, said method comprising:
ii) performing cyclic voltammetry on a plurality of tagged analytes where each species of tagged analyte is tagged with a redox-active label where the redox-active label attached to each species has an oxidation state different and distinguishable from the oxidation states of the redox-active labels attached to the other species of tagged analyte, and said voltammetry produces a cyclic voltogram for said tagged analytes, wherein said cyclic voltammetry is performed by a potentiostat under control of said computer system; and
ii) detecting the signal for a redox-active label at a phase angle out of phase with the optimum phase angle for said redox-active label, where a drop-out of signal at said phase angle indicates the presence of said redox-active label, wherein said detecting comprises analysis of said voltogram by said computer system.

67. A computer system, for detecting a redox active tag among a plurality of redox active tags, said computer system comprising:
a memory configured to store software programs;
a data acquisition and control interface for acquiring data from a potentiostat; and
a computer readable medium comprising computer readable program code for directing said potentiostat in a cyclic voltammetric measurement to produce a cyclic voltammogram of said redox-active tags; and
computer readable program code for detecting the signal for each redox-active label at a phase angle out of phase with the optimum phase angle for said redox-active label, where a drop-out of signal at said phase angle indicates the presence or amount of said redox-active label.


SEQUENCE LISTING (1 sequence)

SEQ ID NO: 1 - DNA, 20 bp, Artificial Sequence
  Primer
aattaaccctcactaaaggg